(19) United States
(12) Patent Application Publication
(10) Pub. No.: US 2024/0153625 A1
(43) Pub. Date: May 9, 2024

(54) BIOMETRIC DATA EVALUATION SERVER, BIOMETRIC DATA EVALUATION SYSTEM, AND BIOMETRIC DATA EVALUATION METHOD

(71) Applicant: LOGISTEED, LTD., Tokyo (JP)

(72) Inventors: Shunsuke MINUSA, Tokyo (JP); Takeshi TANAKA, Tokyo (JP); Hiroyuki KURIYAMA, Tokyo (JP); Kiminori SATO, Tokyo (JP)

(73) Assignee: LOGISTEED, LTD., Tokyo (JP)

(21) Appl. No.: 18/282,308

(22) PCT Filed: Nov. 25, 2021
(86) PCT No.: PCT/JP2021/043239

§ 371 Date: Sep. 15, 2023

(30) Foreign Application Priority Data
Mar. 25, 2021 (JP) 2021-052246

(51) Int. Cl. G16H 40/63; A61B 5/00; A61B 5/024

(52) U.S. Cl. CPC G16H 40/63; A61B 5/02405; A61B 5/746

(57) ABSTRACT

It is provided a biometric data evaluation server including a processor and memory for evaluating biometric data, the biometric data evaluation server comprising: a data collection module configured to receive beat-to-beat interval equivalent data from the biometric data on a subject; and a Lorenz plot generation module configured to calculate a Lorenz plot from the beat-to-beat interval equivalent data at a predetermined period, and output the obtained Lorenz plots as an aggregate Lorenz plot.

### CLAIM OF PRIORITY

The present application claims priority from Japanese patent application JP 2021-52246 filed on May 25, 2021, the content of which is hereby incorporated by reference into this application.

### BACKGROUND OF THE INVENTION

This invention relates to a biometric data evaluation server, system, and method that are used for evaluating reliability of biometric data measured in promoting safe operation of transportation facilities.

In recent years, quantitative evaluation of biological conditions of drivers in the transportation business has been conducted for prevention of accidents caused by health of the drivers. Of the biological conditions, beat-to-beat interval (BBI) data on an interval between heartbeats is measured by various forms of heartbeat sensors that are easy to use for measurement, and autonomic nerve function evaluation is carried out based on the measurement.

For example, in JP 2008-539017 A, there is disclosed a medical device capable of creating a Lorenz plot (LP), which is described later, regarding R-wave interval (R-R Interval or RRI) data of heartbeat data continuously acquired during a predetermined period and detecting arrhythmia by discriminating atrial fibrillation and atrial tachycardia, which are types of arrhythmia, through use of a feature amount relating to the created Lorenz plot.

### SUMMARY OF THE INVENTION

In JP 2008-539017 A, consideration has not been given to a case in which beat-to-beat interval data is irregularly missing in a large number of segments within a predetermined period due to occurrence of poor measurement states or communication failures when the beat-to-beat interval data is measured under a state that is not necessarily a resting state, such as during work involving driving.

There has been a problem in that a degree of occurrence of an arrhythmia-like abnormal value cannot be quantified in a case of partially lacking or missing beat-to-beat interval data.

Therefore, this invention has been made in view of the above-mentioned problem, and has an object to enable evaluation of a degree of occurrence of an arrhythmia-like abnormal value even for beat-to-beat interval data involving lacking of data.

In accordance with at least one embodiment of the present invention, there is provided a biometric data evaluation server including a processor and memory for evaluating biometric data, the biometric data evaluation server comprising: a data collection module configured to receive beat-to-beat interval equivalent data from the biometric data on a subject; and a Lorenz plot generation module configured to calculate a Lorenz plot from the beat-to-beat interval equivalent data at a predetermined period, and output the obtained Lorenz plots as an aggregate Lorenz plot.

Therefore, in accordance with the at least one embodiment of this invention, the degree of occurrence of an arrhythmia-like abnormal value can be evaluated from the generated Lorenz plot even for the beat-to-beat interval data involving the lacking or missing of data. This enables evaluation of reliability of an autonomic nerve function index obtained from the beat-to-beat interval data measured during work.

The details of at least one embodiment of a subject matter disclosed herein are set forth in the accompanying drawings and the following description. Other features, aspects, and effects of the disclosed subject matter become apparent from the following disclosure, drawings, and claims.

### BRIEF DESCRIPTION OF THE DRAWINGS

FIGS. 1A and 1B are block diagrams for illustrating an example of a main configuration of a biometric data evaluation system in accordance with a first embodiment of this invention.

FIG. 2 is a flow chart for illustrating an example of RRI data transmission processing performed by a driving data collection device in accordance with the first embodiment of this invention.

FIG. 3 is a flow chart for illustrating an example of learning processing of a chronic occurrence discrimination model for an arrhythmia-like abnormal value performed by a driving data collection device in accordance with the first embodiment of this invention.

FIG. 4A is a diagram for illustrating an example of a mask region definition used as a feature extraction model by a biometric data evaluation server

FIG. 4B is a diagram for illustrating an example of a poor-measurement-like mask used by a biometric data evaluation server in accordance with the first embodiment of this invention.

FIG. 4C is a diagram for illustrating an example of a normal range mask used by a biometric data evaluation server in accordance with the first embodiment of this invention.

FIG. 4D is a diagram for illustrating an example of a PVC-like abnormal value mask used by a biometric data evaluation server in accordance with the first embodiment of this invention.

FIG. 4E is a diagram for illustrating an example of a missed one-beat detection mask used by a biometric data evaluation server in accordance with the first embodiment of this invention.

FIG. 4F is a diagram for illustrating an example of a missed two-consecutive-beat detection mask used by a biometric data evaluation server in accordance with the first embodiment of this invention.

FIG. 5 is a flow chart for illustrating an example of processing for evaluating the degree of chronic occurrence of an arrhythmia-like abnormal value, which is performed by the biometric data evaluation server in accordance with the first embodiment of this invention.

FIG. 6 is a flow chart for illustrating an example of processing for determining a driver inadequate for the autonomic nerve function evaluation, which is performed by the biometric data evaluation server in accordance with the first embodiment of this invention.

FIG. 7 is a flow chart for illustrating an example of processing for calculating an autonomic nerve function index, which is performed by the biometric data evaluation server in accordance with the first embodiment of this invention.

FIG. 8 is a diagram for illustrating an example of an autonomic nerve function evaluation result screen in accordance with the first embodiment of this invention.

FIG. 9 is a diagram for illustrating an example of a detailed analysis screen for reliability of the RRI data measured during the period A in accordance with the first embodiment of this invention.

FIG. 10A is a diagram for illustrating an example of a data structure of the period-B-unit RRI data in accordance with the first embodiment of this invention.

FIG. 10B is a table for illustrating an example of a data structure of the period-B-unit LP data in accordance with the first embodiment of this invention.

FIG. 10C is a table for illustrating an example of a data structure of the period-A-unit aggregate LP data in accordance with the first embodiment of this invention.

FIG. 10D is a diagram for illustrating an example of a data structure of the aggregate LP feature amount data in accordance with the first embodiment of this invention.

FIG. 10E is a diagram for illustrating an example of a data structure of the abnormality degree data in accordance with the first embodiment of this invention.

FIG. 10F is a diagram for illustrating an example of a data structure of the period-A-unit abnormality discrimination data in accordance with the first embodiment of this invention.

FIG. 10G is a diagram for illustrating an example of a data structure of the inadequacy determination data in accordance with the first embodiment of this invention.

FIG. 10H is a diagram for illustrating an example of a data structure of the autonomic nerve function index data in accordance with the first embodiment of this invention.

FIG. 10I is a diagram for illustrating an example of a data structure of the work state data in accordance with the first embodiment of this invention.

FIG. 10J is a diagram for illustrating an example of a data structure of the history data in accordance with the first embodiment of this invention.

FIG. 10K is a diagram for illustrating an example of a data structure of the chronic occurrence ground truth data in accordance with the first embodiment of this invention.

FIG. 11 is a diagram for illustrating an example relating to definitions of the period A and the period B and occurrence of data lacking in accordance with the first embodiment of this invention.

FIGS. 12A and 12B are block diagrams for illustrating an example of a main configuration of a biometric data evaluation system in a case of also predicting a risk of a traffic accident or incident for a driver through use of the autonomic nerve function index data in accordance with a second embodiment of this invention.

FIG. 13A is a flow chart for illustrating an example of processing for predicting the accident risk during work, which is performed by the biometric data evaluation server in accordance with the second embodiment of this invention.

FIG. 13B is a flow chart for illustrating an example of processing for issuing an alert for warning about an increase in accident risk, which is performed by the biometric data evaluation server in accordance with the second embodiment of this invention.

FIG. 14 is a diagram for illustrating an example of a warning presentation screen to be issued to the driver when an increase in accident risk is detected in accordance with the second embodiment of this invention.

### DETAILED DESCRIPTION OF THE PREFERRED EMBODIMENTS

Now, embodiments of this invention are described with reference to the accompanying drawings.

### First Embodiment

First, a first embodiment of this invention is described.

### <System Configuration>

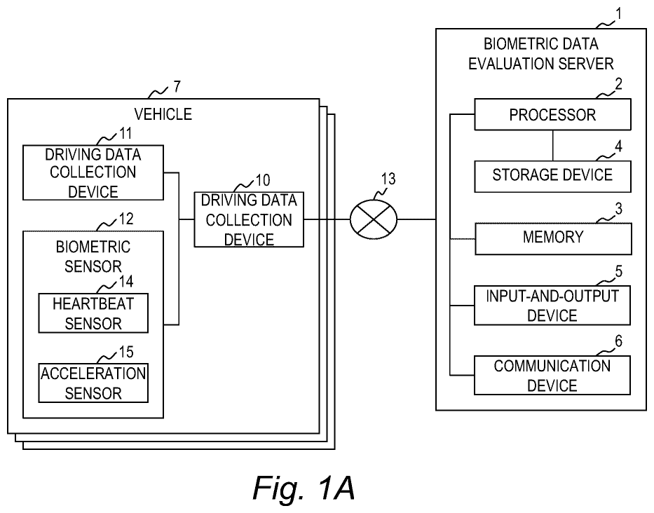

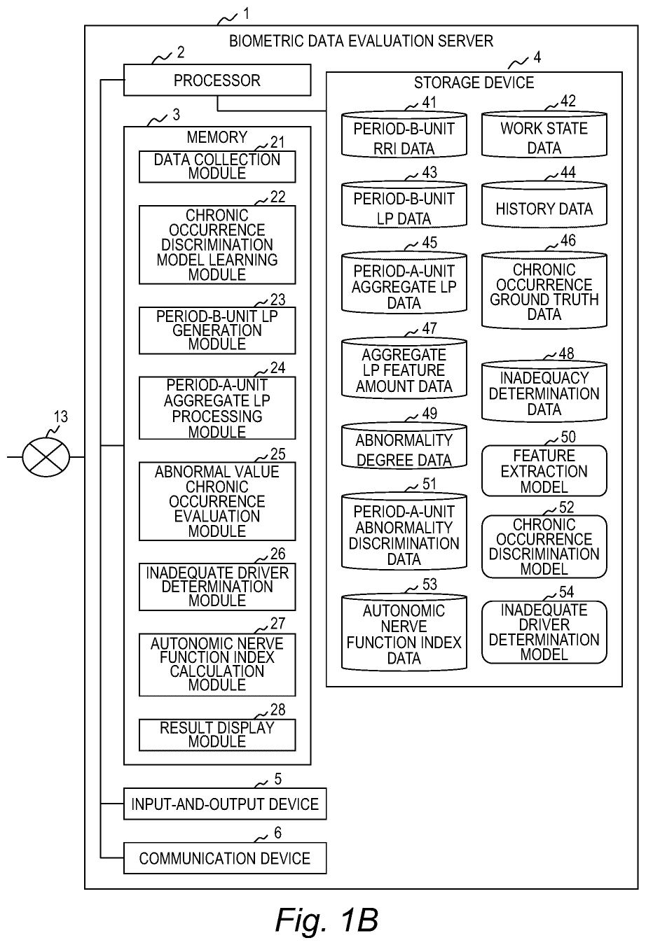

FIGS. 1A and 1B are block diagrams for illustrating an example of a main configuration of a biometric data evaluation system in accordance with the first embodiment of this invention. The biometric data evaluation system in accordance with the first embodiment includes a biometric data evaluation server 1 that processes data received from one or more vehicles 7 through a network 13.

The vehicle 7 includes a biometric sensor 12 that detects biometric data on a driver, a driver ID reading device 11 that detects a driver ID for identifying the driver, and a driving data collection device 10 that collects the detected biometric data and the driver ID and transmits the biometric data and the driver ID to the biometric data evaluation server 1.

The biometric sensor 12 includes a heartbeat sensor 14 that detects an RRI and an acceleration sensor 15 that detects movement of the driver. As the heartbeat sensor 14, it is possible to use a sensor that detects a heartbeat based on an electrocardiogram, pulse waves, heart sound, or the like.

The biometric sensor 12 is not limited to the above-mentioned example, and not only the heartbeat sensor 14 but also a sensor that detects an amount of perspiration, a body temperature, blinking, eye movement, myoelectricity, an electroencephalogram, or the like can be adopted. As the biometric sensor 12, it is possible to use, for example, a wearable device that can be worn by the driver as well as a sensing device attached to the inside of a vehicle such as a steering wheel, a seat, or a seat belt, or an image recognition system that picks up an image of a facial expression and behavior of the driver and analyzes the image.

The heartbeat sensor 14 may detect a beat-to-beat interval other than an RRI, such as a PPI, which is a pulse wave interval. In another case, it is also possible to obtain beat-to-beat interval equivalent data by estimating a beat-to-beat interval from image data on a face of the driver. In the first embodiment, any biometric data that enables calculation of a beat-to-beat interval may be used, and can include such beat-to-beat interval equivalent data as described above.

The driver ID reading device 11 reads a card on which an identifier of the driver is recorded. The driving data collection device 10 collects data from the biometric sensor 12 at a predetermined cycle, and transmits the data to the biometric data evaluation server 1 through the network 13.

In the example illustrated in FIG. 1A, the driver ID reading device 11 is configured as a device that reads the card on which the identifier of the driver is recorded, but may be configured in a different manner. For example, when the driver ID reading device 11 is one mobile terminal and the mobile terminal is caused to function as a driver ID reading unit, the driver ID reading device 11 may read the driver ID by causing the driver himself or herself to input the identifier of the driver, or may read the driver ID by identifying the driver by a publicly known face authentication technology using a camera included in the mobile terminal.

The biometric data evaluation server 1 is a computer including a processor 2, a memory 3, a storage device 4, an input-and-output device 5, and a communication device 6. The memory 3 loads, as a program, each of functional modules including a data collection module 21, a chronic occurrence discrimination model learning module 22, a period-B-unit LP generation module 23, a period-A-unit aggregate LP processing module 24, an abnormal value chronic occurrence evaluation module 25, an inadequate driver determination module 26, an autonomic nerve function index calculation module 27, and a result display module 28. Each program is executed by the processor. Details of the respective functional modules are described later.

The processor 2 operates as a functional module that provides a predetermined function by executing processing in accordance with the program of each functional module. For example, the processor 2 functions as the abnormal value chronic occurrence evaluation module 25 by executing an abnormal value chronic occurrence evaluation program. The same applies to other programs. In addition, the processor 2 also operates as a functional module that provides each of functions of a plurality of processes executed by each program. The computer and computer system are the device and system that include those functional modules.

The storage device 4 stores data to be used by each of the above-mentioned functional modules. The storage device 4 stores period-B-unit RRI data 41, period-B-unit LP data 43, period-A-unit aggregate LP data 45, aggregate LP feature amount data 47, abnormality degree data 49, period-A-unit abnormality discrimination data 51, autonomic nerve function index data 53, work state data 42, history data 44, chronic occurrence ground truth data 46, inadequacy determination data 48, a feature extraction model 50, a chronic occurrence discrimination model 52, and an inadequate driver determination model 54. The “LP” is an abbreviation for “Lorenz plot” (hereinafter also referred to as “LP”; and also called “Poincare plot”), and the same notation is used below.

The input-and-output device 5 includes an input device such as a mouse, a keyboard, a touch panel, or a microphone and an output device such as a display or a speaker. The communication device 6 communicates to/from the vehicle through the network 13.

In the first embodiment, a case in which biometric data measured from the driver who operates the vehicle 7 is used is described as an example, but a form of the biometric data is not limited to the first embodiment. For example, instead of the driver who operates the vehicle 7, the subject may be a driver who operates a moving object such as an airplane or a train. In addition to the drivers, other examples of the subject may also include: general employees who are not limited to the drivers who have biometric data measured during work; or people who lead daily lives that are not limited to working hours.

### <Details of Processing>

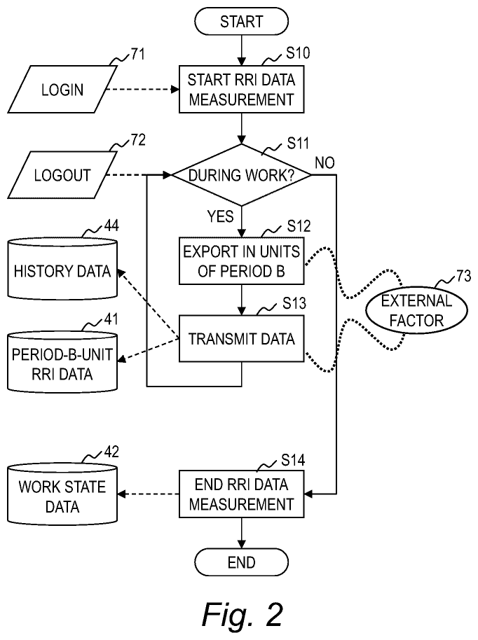

FIG. 2 is a flow chart for illustrating an example of RRI data transmission processing performed by the driving data collection device 10 of the vehicle. In the first embodiment, a case in which a first period A (window width) being a subject period for evaluating a degree of chronic occurrence of an arrhythmia-like abnormal value is set to, for example, one work day and a second period B having a window width equal to or smaller than that of the period A is set to, for example, a unit of 2 minutes is described as an example.

The period A and the period B may be determined by a segment that defines a duration as in the first embodiment, or may be determined by the number of data points. For example, the period B may be set to 120 data points while the period A is set to one work day in the same manner.

Further, the period B is preferred to be equal to or shorter than the first period A and equal to or longer than the duration required for heart rate variability analysis. The heart rate variability analysis is difficult based on only one beat, and requires about 10 seconds. The period B may also be determined based on information on the duration of the lacking or missing of RRI data received from the input device.

First, when the driving data collection device 10 receives a login input 71 from the driver, the driving data collection device 10 starts RRI data measurement (Step S10). For example, as the login input 71, an event in which the card on which the identifier of the driver is recorded is read through use of the driver ID reading device 11 may be used.

Next, the driving data collection device 10 exports (Step S12) and transmits (Step S13) the measured RRI data until Step 511 of receiving a logout input 72 from the driver. In the first embodiment, a case in which, on the driving data collection device 10 side of the vehicle 7, the measured RRI data is divided and exported in units of the period B in advance for each period B irrespective of a measurement status and is sequentially transmitted to the biometric data evaluation server 1 in units of the period B is described. On the biometric data evaluation server 1, the data collection module 21 receives the RRI data and stores the RRI data in the period-B-unit RRI data 41.

Instead of transmitting the RRI data divided in units of the period B in advance on the driving data collection device 10 side, for example, the period-A-unit RRI data may be measured and transmitted to the biometric data evaluation server 1 in units of the period A, and then the data collection module 21 of the biometric data evaluation server 1 may divide the period-A-unit RRI data in units of the period B and store the period-A-unit RRI data in the period-B-unit RRI data 41.

Further, instead of always transmitting the RRI data divided in units of the period B for each period B, the driving data collection device 10 may monitor the measurement status and, only when a lack of RRI data satisfies a certain condition, may cut out the RRI data in units of the period B to transmit the RRI data to the biometric data evaluation server 1.

When the RRI data relating to the period B has been measured, the driving data collection device 10 executes processing (Step S12) for exporting the RRI data in a data transmission format in units of the period B. After the period-B-unit exporting processing (Step S12) is ended, the driving data collection device 10 transmits the exported period-B-unit RRI data 41 to the biometric data evaluation server 1, and stores measurement information relating to the period-B-unit RRI data 41 in the history data 44 of the biometric data evaluation server 1.

The driving data collection device 10 executes the above-mentioned processing in units of the period B until the driving data collection device 10 receives the logout input 72 from the driver (Step S11). For example, an event timer may be provided in the driving data collection device 10, to thereby count a lapse of two minutes, which are an example of the period B, and execute Step S11.

Further, as the logout input 72, an event in which the card on which the identifier of the driver is recorded is read through use of the driver ID reading device 11 for the first time after the login input 71 was performed may be used.

The history data 44 stores information relating to each piece of data of the period-B-unit RRI data 41 measured through the driving data collection device 10. For example, a driver ID and a vehicle ID for associating the period-B-unit RRI data 41 with the driver and the vehicle 7, respectively, are stored. Further, a time at which the data was transmitted to the biometric data evaluation server 1 and a transmitted file name may be stored. In addition, a state of the driver during the period B may be stored. The state of the driver is information indicating under what kind of measurement condition the period-B-unit RRI data 41 was measured. For example, classifications of “driving” and “stopped” obtained by the biometric sensor 12 may be stored.

The state of the driver is not required to be explicitly stored in the history data 44. For example, in a configuration in which the driving data collection device 10 performs the period-B-unit exporting processing of Step S12 only while the vehicle is traveling and the RRI data measured while the vehicle is not traveling is discarded, it is obvious that all pieces of period-B-unit RRI data to be transmitted to the biometric data evaluation server 1 were measured while the vehicle was traveling, and hence it is not required to store the state of the driver.

In this case, a partial segment (period B) of the period-A-unit RRI data is lacking due to an external factor 73 in the period-B-unit exporting processing (Step S12) and the data transmission processing (Step S13).

In the first embodiment, the example in which the period-B-unit exporting processing occurs every two minutes irrespective of the measurement status has been described, and there occurs a case in which, as a result of not detecting an RRI due to poor measurement, empty period-B-unit RRI data 41 is generated, that is, there is a lack of RRI data. Further, for example, there occurs a case in which the transmission processing may fail due to a communication failure or the like in the data transmission processing (Step S13), thereby causing a lack of the period-B-unit RRI data 41.

As described above, a lack in the period-A-unit RRI data is, for example, caused when RRI data is missing due to absence of the period-B-unit RRI data 41 or caused by the period-B-unit RRI data 41, irrespective of presence thereof, as a result of having a considerably small amount of valid RRI data or being empty.

When the driving data collection device 10 receives the logout input 72, the driving data collection device 10 advances the process to Step S14 of RRI data measurement ending processing. In the RRI data measurement ending processing (Step S14), the work state data 42 including work information on the driver relating to the period A is generated and transmitted to the biometric data evaluation server 1.

When there is no valid RRI data and the empty period-B-unit RRI data 41 is generated, the subsequent data transmission processing (Step S13) may be skipped.

In accordance with the above-mentioned processing, after the driving data collection device 10 starts to measure RRI data, the RRI data is transmitted to the biometric data evaluation server 1 in units of the period B, and the period-B-unit RRI data 41 is accumulated in the biometric data evaluation server 1 for each driver.

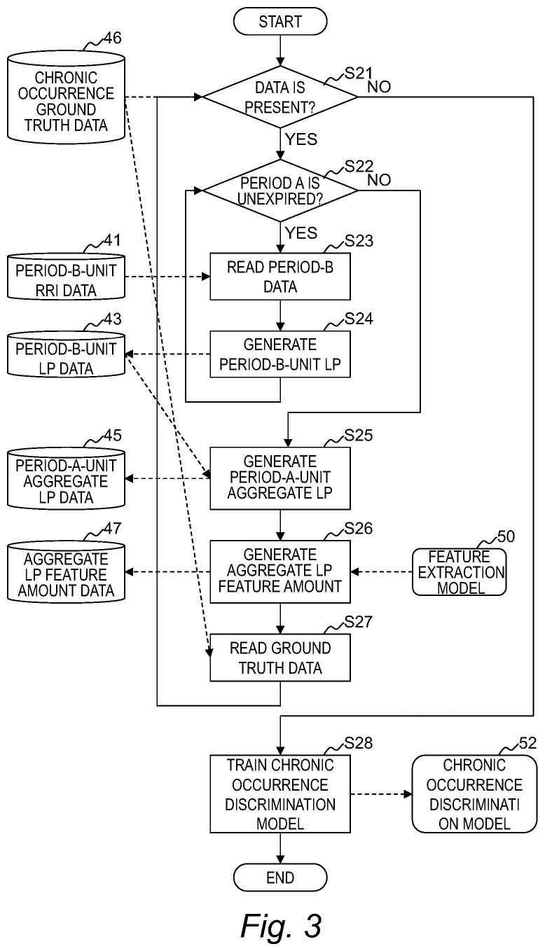

FIG. 3 is a flow chart for illustrating an example of learning processing of the chronic occurrence discrimination model 52 for an arrhythmia-like abnormal value, which is performed by the biometric data evaluation server 1. First, the chronic occurrence discrimination model learning module 22 refers to the chronic occurrence ground truth data 46, which defines whether or not an arrhythmia-like abnormal value is chronically occurring in the RRI data measured during a certain period A, to determine presence or absence of the chronic occurrence ground truth data 46 to be learned (Step S21).

When the chronic occurrence ground truth data 46 to be learned is present, the chronic occurrence discrimination model learning module 22 advances the process to Step S22 to perform the subsequent processing on each period A stored in the chronic occurrence ground truth data 46. The chronic occurrence ground truth data 46 to be learned can be set in advance through the input-and-output device 5.

The chronic occurrence discrimination model learning module 22 generates, for each period A stored in the chronic occurrence ground truth data 46, the aggregate LP feature amount data 47 to be used for evaluating whether or not an arrhythmia-like abnormal value is chronically occurring in the period-B-unit RRI data 41 measured during the period A.

As described above, the aggregate LP feature amount data 47 is data to be input to the chronic occurrence discrimination model 52 and used for determining whether or not the arrhythmia-like abnormal value (abnormal value of a feature amount of arrhythmia) is chronic.

First, the chronic occurrence discrimination model learning module 22 calls the period-B-unit LP generation module 23, and the period-B-unit LP generation module 23 reads the period-B-unit RRI data 41 obtained by dividing the RRI data within the period A in units of the period B (Step S23).

At a time of reading the period-B-unit RRI data 41, in consideration of a case in which the read period-B-unit RRI data 41 has a considerably small amount of valid RRI data or is empty, the period-B-unit LP generation module 23 may inspect the subject period-B-unit RRI data 41 to determine presence or absence of a lack of data. In this case, for the period-B-unit RRI data 41 determined to correspond to a lack of data, the subsequent processing is skipped, and the period B is handled as data lacking.

Further, the period-B-unit LP generation module 23 may additionally read the history data 44 at the time of reading the period-B-unit RRI data 41. In this case, the period-B-unit LP generation module 23 may refer to the state of the driver stored in the history data 44 to cancel the reading of the period-B-unit RRI data 41 when the read state does not match a predetermined state of the driver and skip the subsequent processing relating to the applicable data.

For example, when the state of the driver is not “driving,” the applicable period-B-unit RRI data 41 may be excluded from the learning processing for a chronic occurrence discrimination model without being read. In this case, the measurement condition for the period-B-unit RRI data 41 to be used for the learning of the chronic occurrence discrimination model 52 is unified, and evaluation accuracy of an arrhythmia-like abnormal value under the measurement condition is expected to improve.

Subsequently, the period-B-unit LP generation module 23 uses the period-B-unit RRI data 41 read as described above to generate a Lorenz plot in units of the period B, and stores the Lorenz plot as the period-B-unit LP data 43 (Step S24).

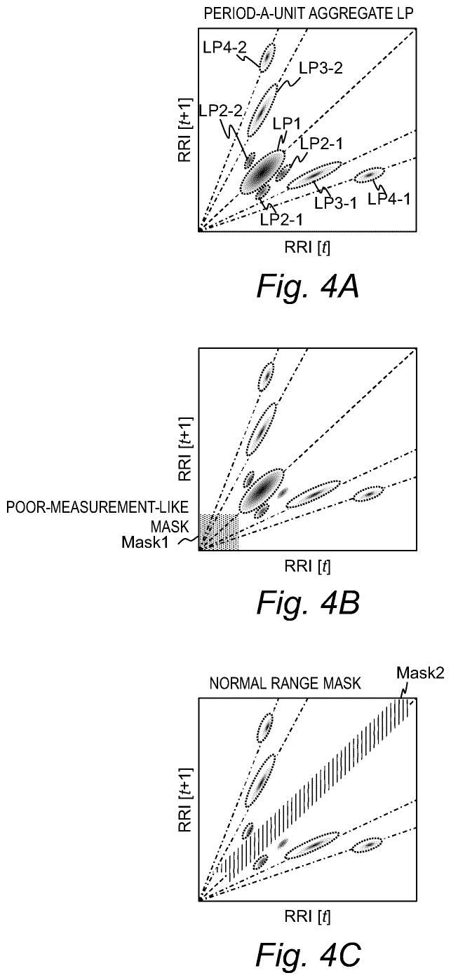

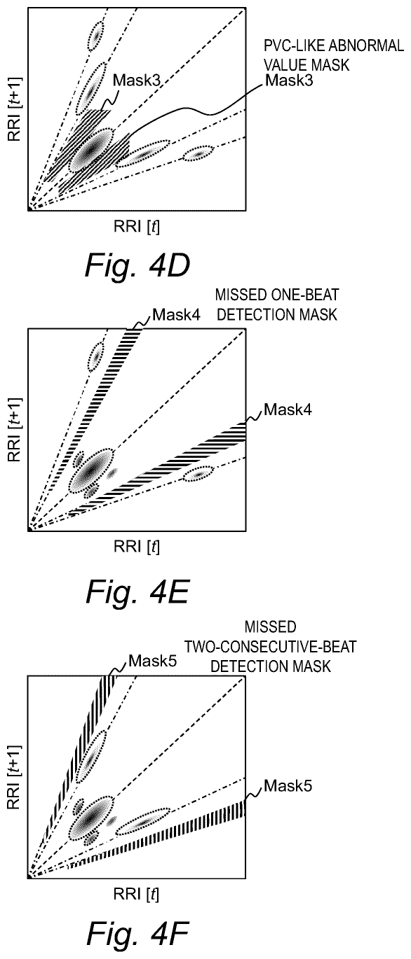

The LP is a two-dimensional plot used for subjecting time-series data T[t] to chaos analysis and drawn in FIG. 4A by plotting time-series data T[t] for a preceding time t (t=0, 1, 2, . . . ) on one axis thereof and time-series data T[t+dt] for a time t+dt, which is later than the time t by a period dt (dt=1, 2, 3, . . . ) on another axis thereof.

In the first embodiment, an LP obtained through plotting by associating T[t] with an x-axis and T[t+dt] with a y-axis is described as an example. Characteristics of the time-series data T[t] can be analyzed by analyzing a geometric figure drawn on the LP.

In particular, it is known that, when an LP regarding the RRI data in consecutive pieces of period-B-unit RRI data is drawn, measurement characteristics such as how much arrhythmia and poor measurement are included in the data measured during the period B can be evaluated from geometric features on the LP.

In the first embodiment, an example of the LP in which dt is set to a time one beat after t is described. In addition, a case in which an original series RRI[t] of the measured RRI data is used as the time-series data is described. It should be noted that dt may be larger than 1, that is, two points temporally adjacent to each other are not required to be used. Further, the RRI data to be used is not limited to the original series RRI[t], and, for example, a difference series ΔRRI[t] obtained by taking a difference between adjacent data points may be used.

In the first embodiment, in order to quantitatively evaluate the geometric features on the LP, the period-B-unit LP generation module 23 calculates an LP matrix that expresses an appearance frequency of each data point on the LP as density (or brightness). As described later with reference to FIG. 4A, the density becomes higher as the appearance frequency becomes higher, and the density becomes lower as the appearance frequency becomes lower. For example, in the first embodiment, the x-axis and the y-axis are each set to have a range of from 0 milliseconds to 2,560 milliseconds, and mesh regions obtained by drawing a grid square so as to divide the range into 64 in each axis direction is defined.

In other words, in this case, each mesh represents a region having a mesh width of 40 milliseconds and an area of (40 milliseconds)×(40 milliseconds). Then, the number of data points on the LP belonging to each mesh is counted, and the density of each mesh region is defined as a component of the LP matrix. The drawing range of the LP and the division amount of each axis, that is, the mesh width are not limited to those described in the first embodiment. For example, the mesh regions may be defined so as to have the same drawing range and an 80-millisecond-unit mesh width, that is, each axis being divided into 32.

In addition, in order to clearly evaluate measurement characteristics exhibited when the RRI data is not measured normally, in the first embodiment, an example of adopting, as pre-processing for LP generation, saturation processing for setting a threshold value for the density of a mesh region and replacing the density exceeding the threshold value by the threshold value is described.

For example, with the threshold value being set to three times, the density has a maximum value of three times even when data appears in the mesh region four or more times. This processing emphasizes the geometric features of arrhythmia-like and poor-measurement-like RRI abnormal values.

With the above, in the first embodiment, the period-B-unit LP generation module 23 generates, as the period-B-unit LP data 43, an LP matrix of 64×64 having a density with four gray levels from 0 to 3.

The pre-processing for the LP generation is not limited to the saturation processing. For example, in order to process only an arrhythmia-like abnormal value by this method, an RRI having a length of about n times as large as the assumed RRI ascribable to n-consecutive-beat R-wave misdetection may be simultaneously subjected to abnormal value exclusion or replacement processing using a median filter, which is a publicly known time domain analysis method, misdetected beat interpolation processing using a Kalman filter, which is also a publicly known method, or the like.

The chronic occurrence discrimination model learning module 22 executes the above-mentioned processing for calculating the period-B-unit LP data 43 from the period-B-unit RRI data 41, which has been obtained through division in units of the period B, for all the periods B included in the period A (Step S22). Even when the period B is included in the period A, the period B that does not exist as the period-B-unit RRI data 41 is handled as data lacking.

After that, the chronic occurrence discrimination model learning module 22 performs period-A-unit aggregate LP generation processing (Step S25) for generating the period-A-unit aggregate LP data 45 by aggregating the obtained period-B-unit LP data 43 in units of the period A.

In this flow chart, the example of sequentially executing the generation of the period-B-unit LP data 43 over the period A is illustrated, but the generation may be executed in parallel instead of being executed sequentially.

In a case in which the RRI data has been continuously measured in time series during the period B or in which the RRI data corresponding to the period B can be obtained by the abnormal value replacement, interpolation, or exclusion processing, an LP generated from the RRI data for the period B has been hitherto researched, and there is a large amount of knowledge about what kind of geometrical shape corresponds to an arrhythmia-like abnormality.

Meanwhile, in regard to the period A having a window width larger than that of the period B, an RRI data amount actually measured during the period A varies due to various factors such as a time slot in which an RRI cannot be continuously measured, a time slot in which there is no RRI data due to a failure in measurement, a time slot in which the RRI data was present but has failed to be transmitted to a server to cause a lack of data, and a case in which the start and end of one work day, which is described as an example of the period A, differ for some reason of the work.

For example, when there are 200 segments obtained by simply dividing the period A by the period B, there may be a case in which the measurement was performed during only 190 segments of periods B within the period A in which the measurement was actually performed. The LP generation performed when RRI data is thus lacking in a segment within the period A has not been taken into consideration in the related art, and geometric features to be shown in such a case have not been clear.

In the period-A-unit aggregate LP generation processing (Step S25), the chronic occurrence discrimination model learning module 22 calls the period-A-unit aggregate LP processing module 24, and the period-A-unit aggregate LP processing module 24 generates the period-A-unit aggregate LP data 45, which is an LP that quantifies the feature amount of the RRI data corresponding to the period A, by performing the aggregation processing (Step S25) on a period-B-unit LP data 43 group corresponding to the periods B that are present in the period A to be subjected to the evaluation, that is, are not data lacking periods.

The period-A-unit aggregate LP processing module 24 generates the period-A-unit aggregate LP data 45 by, for example, performing averaging processing on the period-B-unit LP data 43 group for each component of the LP matrix. In the case of the first embodiment, the period A is one work day, and hence the period-A-unit aggregate LP processing module 24 generates a work-day-unit average LP, and stores the work-day-unit average LP as the period-A-unit aggregate LP data 45.

A method for the aggregation processing is not limited to the averaging processing for each component of the LP matrix. For example, in order to improve robustness during the aggregation processing, the aggregation processing may be performed by statistical processing such as calculating an N-th percentile within the period A for each component of the LP matrix.

In this case, when the aggregation processing with N=50% is selected, a median value for each component is used for the aggregation processing, and when the period-B-unit LP data 43 having characteristics noticeably different from others is present within the period A, it is possible to perform the aggregation processing in which an influence of the above-mentioned data having the different characteristics is reduced compared to the averaging processing.

In a case in which there is a lack of RRI data in a segment within the period A, when an LP is simply generated through use of all pieces of RRI data, there are a case in which processing for emphasizing an RRI abnormal value based on a density threshold value does not exhibit a desired function and a case in which geometric features that are essentially supposed to appear on the LP are no longer observed.

Meanwhile, when this method is used to generate an LP for each of the periods B in which no lack of RRI data occurs and aggregate the LPs in units of the period A, the knowledge relating to the period B which has been hitherto obtained through research can be applied to the period A including a lack of RRI data.

In the case of the first embodiment, in the period-A-unit aggregate LP generation processing (Step S25), the period-A-unit aggregate LP data 45 having a density represented by the consecutive values from 0 to 3 is generated from the period-B-unit LP data 43 created with four gray levels from 0 to 3 in the first embodiment.

Subsequently, the chronic occurrence discrimination model learning module 22 calls the period-A-unit aggregate LP processing module 24, and the period-A-unit aggregate LP processing module 24 uses the feature extraction model 50, which is a model for extracting the aggregate LP feature amount data 47 indicating a degree of an arrhythmia-like abnormality or a poor-measurement-like abnormality from the period-A-unit aggregate LP data 45, to perform aggregate LP feature amount generation processing (Step S26).

The feature extraction model 50 is formed of analysis means for extracting a feature of a two-dimensional matrix. It is possible to use, for example: a mask-pattern-based feature extraction method of extracting, with the two-dimensional matrix of the period-A-unit aggregate LP data 45 being regarded as an image, data density or the appearance frequency as a feature amount from a mask region group defined in advance for extracting geometric features by being created based on prior knowledge relating to the arrhythmia-like abnormality or the poor-measurement-like abnormality; a convolutional-neural-network-based model which has learned information relating to the arrhythmia-like abnormality or the poor-measurement-like abnormality as ground truth data based on the prior knowledge; and a Fourier-transform-based frequency domain analysis model. In the first embodiment, the mask-pattern-based feature extraction method is described in detail with reference to FIG. 4A to FIG. 4F.

The period-B-unit LP generation module 23 may additionally read the history data 44 at the time of reading the period-B-unit RRI data 41 in Step S23, and when only data that matches a predetermined driver state is used, the feature extraction model 50 suitable for the predetermined driver state may be used. For example, the measurement characteristics of the RRI data change due to a difference in behavior of the driver between when the driver state is “driving” and when the driver state is “stopped,” and hence appropriate feature extraction is expected through selective use of the feature extraction model 50.

After that, the chronic occurrence discrimination model learning module 22 reads the chronic occurrence ground truth data 46 corresponding to the period A (Step S27). The chronic occurrence ground truth data 46 stores whether or not an arrhythmia-like abnormal value is chronically occurring during a certain period A through use of, for example, 0 and 1. The above-mentioned processing steps are performed on the chronic occurrence ground truth data 46 group to be learned.

After the chronic occurrence ground truth data 46 and the aggregate LP feature amount data 47 corresponding thereto have been generated, the chronic occurrence discrimination model learning module 22 performs chronic occurrence discrimination model learning processing (Step S28) for generating the chronic occurrence discrimination model 52 from the aggregate LP feature amount data 47.

The chronic occurrence discrimination model 52 can be formed through use of a publicly known discrimination algorithm. Examples to be used as a machine learning algorithm of the discrimination algorithm can include a logistic regression model, a decision tree, a random forest, a support vector machine, a neural network, and a deep learning model.

The chronic occurrence discrimination model 52 can discriminate the presence or absence of chronic occurrence of arrhythmia from the aggregate LP feature amount data 47, and a discrimination probability of the presence or absence of the chronic occurrence can also be used as a calculation model for an abnormality degree indicating the degree of an arrhythmia-like RRI abnormality in the period A.

When the history data 44 is additionally read at the time of reading the period-B-unit RRI data 41, a plurality of chronic occurrence discrimination models 52 may be generated for each state of the driver stored in the history data 44. In this case, a degree of occurrence of an arrhythmia-like abnormal value can be evaluated in consideration of the characteristics of the RRI data corresponding to the measurement condition.

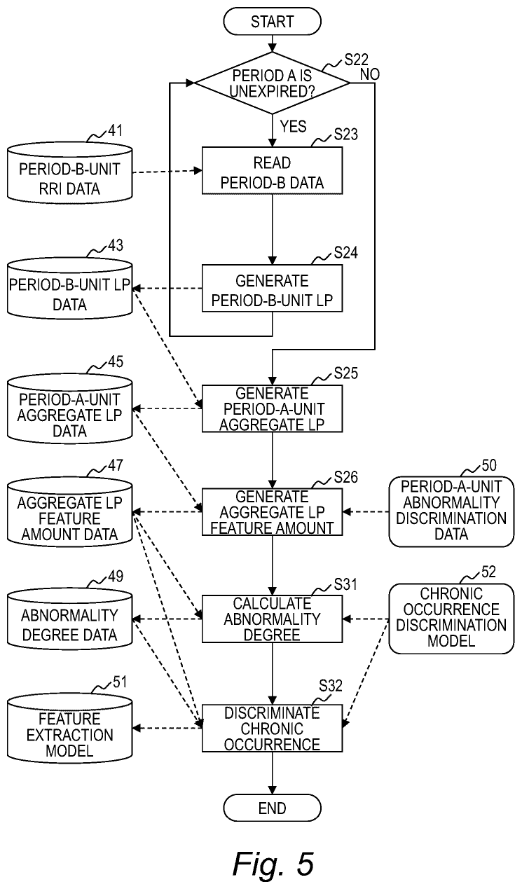

This learning processing is executed at least once prior to processing for evaluating a degree of chronic occurrence of an arrhythmia-like abnormal value, which is described later with reference to FIG. 5. In addition, this processing can be performed at regular intervals in accordance with as an increase of the chronic occurrence ground truth data 46, and the chronic occurrence discrimination model 52 can be trained again. With the above, the chronic occurrence discrimination model 52 having higher discrimination accuracy can be generated.

FIG. 4A to FIG. 4F are graphs for showing examples of mask region definitions for the mask-pattern-based feature extraction method, which are used as the feature extraction model 50 for use in the biometric data evaluation server 1.

In the first embodiment, the period-A-unit aggregate LP data 45 shown in FIG. 4A is taken as an example, and an example of a feature extraction method based on a mask pattern formed of a total of five masks shown in FIG. 4B to FIG. 4F is described. In the mask-pattern-based feature extraction method, the period-A-unit aggregate LP processing module 24 extracts, as feature amounts, values of statistics such as an average or sum of densities inside and outside defined mask regions and values of the number of mesh regions having densities equal to or larger than a fixed threshold value (Step S26 of FIG. 3).

FIG. 4A is an example of the period-A-unit aggregate LP data 45, and densities on LP1 to LP4-2 indicate RRI distributions in the respective mesh regions. In addition, the broken straight lines indicate y=nx (n=3, 2, 1, ½, ⅓), respectively.

In addition, RRI[t] on the horizontal axis indicates an RRI at a certain time, and RRI[t+1] on the vertical axis indicates an RRI at a time of a heartbeat subsequent to the certain time.

FIG. 4B is an example of a mask region (poor-measurement-like mask Mask1) that defines a range in which poor-measurement-like abnormal values in an unstable measurement state which are frequently observed due to factors other than beat detection failures are distributed. The poor measurement ascribable to the measurement instability occurs due to inability to capture peaks of R waves, and hence both R waves and noise are detected as R waves at intervals shorter than an average RRI that is essentially supposed to be measured. In this example, the mask region (Mask1) is defined by Expression (1).

$$Mask 1 ( x , y ) = \{ \begin{matrix}x < a \\ y < b\end{matrix} \qquad ( 1 )$$

For example, when it is assumed that the RRI of the driver rarely falls below 500 milliseconds during driving, a and b can each be set to 500 milliseconds, to thereby be able to quantify a degree of abnormal values assumed to be ascribable to the measurement instability.

FIG. 4C is an example of a mask region (Mask2) that defines a distribution range of RRI data considered to have an RRI fluctuating in a normal range. In this example, the mask region (Mask2) is defined by Expression (2).

$$Mask 2 = ( x , y , Fluct Range ) = \{ \begin{matrix}x - Fluct Range \leq y \leq x + Fluct Range \\ not Mask 1 ( x , y )\end{matrix} \qquad ( 2 )$$

FluctRange is a fluctuation range that can be taken by the RRI data. For example, referring to a variance σ2 of heart rate variability in a state of rest with eyes closed, assuming that the RRI data is widely distributed within 2σ, FluctRange can be set to 80 milliseconds.

An RRI data group of LP1 shown in FIG. 4A is indicated to be included in a region of the normal range mask (Mask2).

Next, a mask region for separating arrhythmia-like abnormal values and poor-measurement-like abnormal values due to missed beat detection to obtain feature amounts is described. The description of the first embodiment is directed to an example in which, of the arrhythmia-like abnormal values, in particular, premature ventricular contraction (PVCs) and premature atrial contractions (PACs) frequently occur.

The poor-measurement-like abnormal values due to the missed beat detection are distributed near y=(n+1)x and y=1/(n+1)x in a case of missed n-beat detection. Meanwhile, the arrhythmia-like abnormal values are distributed in a region from y=x to y=2x and y=½x in a case of premature-ventricular-contraction-like and premature-atrial-contraction-like RRIs. Therefore, for the quantitative evaluation of arrhythmia-like abnormal values, it is effective to evaluate the region from y=x to y=2x and y=½x so as not to include a distribution region of missed one-beat detection.

FIG. 4D is an example of mask regions (Mask3) that define a range in which arrhythmia-like abnormal values are distributed. In this example, the mask regions are defined as follows.

$$Mask 3 ( x , y ) = \{ \begin{matrix}x + Fluct Range < y \leq tan ( {tan}^{- 1} 2 - Δθ ) x … ( a ) \\ x - Fluct Range > y \geq tan ( {tan}^{- 1} ( 1 / 2 ) + Δθ ) x … ( b ) \\ x \leq Max RRI … ( c ) \\ y \leq Max RRI … ( d ) \\ ( ( a ) or ( b ) ) and ( ( c ) and ( d ) ) and ( not Mask 1 ( x , y ) )\end{matrix} \qquad ( 3 )$$

A PVC-like abnormal value mask (Mask3) enables arrhythmia-like RRIs to be evaluated by evaluating regions between y=x±FluctRange and straight lines obtained by relaxing y=2x and y=½x by a rotation angle Δθ about the origin with a counterclockwise direction being set as a positive direction so as not to include the distribution region of the missed one-beat detection described later.

The rotation angle Δθ may be set to, for example, Δθ=2°. In addition, a region of RRIs equal to or larger than an interval MaxRRI, which is an interval considered to be unlikely during driving, is excluded, to thereby be further able to evaluate only arrhythmia-like abnormal values. For example, on the assumption that the RRI data fails to be correctly measured when the heart rate is below 40 during driving, MaxRRI can be set to 1,500 milliseconds.

RRI data groups of LP2-1 and LP2-2 shown in FIG. 4A are indicated to be included in the regions of the PVC-like abnormal value mask (Mask3).

FIG. 4E is an example of mask regions (Mask4) that define a range in which RRI abnormal values are distributed in a case of missed n-consecutive-beat detection (where n=1), and the mask regions (Mask4) are defined as follows.

$$Miss Detect ( x , y ) = \{ \begin{matrix}tan ( {tan}^{- 1} ( n + 1 ) - Δθ ) x < y \leq tan ( {tan}^{- 1} ( n + 2 ) - Δθ ) x … ( a ) \\ tan ( {tan}^{- 1} ( 1 / ( n + 1 ) ) + Δθ ) x > y \geq tan ( {tan}^{- 1} ( 1 / ( n + 2 ) ) + Δθ ) x … ( b ) \\ ( ( a ) or ( b ) ) and ( not Mask 1 ( x , y ) )\end{matrix} \qquad ( 4 )$$

RRI data groups of LP3-1 and LP3-2 shown in FIG. 4A are indicated to be included in the regions of a missed one-beat detection mask (Mask4).

FIG. 4F is an example of mask regions (Mask5) that define a range in which RRI abnormal values due to missed n-consecutive-beat detection (where n=2) are distributed, and the mask regions (Mask5) are defined in the same manner as in FIG. 4E. RRI data groups of LP4-1 and LP4-2 shown in FIG. 4A are indicated to be included in the regions of a missed two-consecutive-beat detection mask (Mask5).

Through extraction of features from a period-A-unit aggregate LP as described above, it is possible to separately evaluate a degree of arrhythmia-like abnormal values and a degree of poor-measurement-like abnormal values. The mask regions are not limited to only those described in the first embodiment. For example, a mask region for expressing atrial fibrillation may be additionally defined and used.

As described above, the feature amount that enables the evaluation by separating poor-measurement-like abnormal values and the mask regions that define arrhythmia-like abnormal values is calculated, thereby producing an effect of enabling the selective evaluation of a chronic occurrence status of an arrhythmia-like abnormal value. Further, as a secondary effect, an effect of similarly enabling the evaluation of the degree of poor-measurement-like abnormal values is obtained.

FIG. 5 is a flow chart for illustrating an example of processing for evaluating the degree of chronic occurrence of an arrhythmia-like abnormal value, which is performed by the biometric data evaluation server 1. In FIG. 5, Step S22 to Step S26 are the same processing steps as those of the flow chart of FIG. 3, and are thus denoted by the same reference symbols as those of FIG. 3.

First, the period-B-unit LP generation module 23 reads the period-B-unit RRI data 41 obtained by dividing the RRI data corresponding to the period A in units of the period B (Step S23). Subsequently, the period-B-unit LP generation module 23 uses the read period-B-unit RRI data 41 to generate an LP in units of the period B, and stores the LP as the period-B-unit LP data 43 (Step S24).

After the period-B-unit LP generation module 23 has generated the period-B-unit LP data 43 for all the periods B included in the period A (Step S22), the period-A-unit aggregate LP processing module 24 performs the period-A-unit aggregate LP generation processing (Step S25) for generating the period-A-unit aggregate LP data 45 obtained by performing the aggregation processing on the period-B-unit LP data 43 in units of the period A.

Then, the period-A-unit aggregate LP processing module 24 uses the feature extraction model 50, which is a model for extracting an aggregate LP feature amount indicating the degree of the arrhythmia-like abnormality or the poor-measurement-like abnormality from the period-A-unit aggregate LP data 45, to perform the aggregate LP feature amount generation processing (Step S26).

After that, the abnormal value chronic occurrence evaluation module 25 subjects the obtained aggregate LP feature amount data 47 to abnormality degree calculation processing (Step S31) for calculating the abnormality degree data 49 through use of the trained chronic occurrence discrimination model 52.

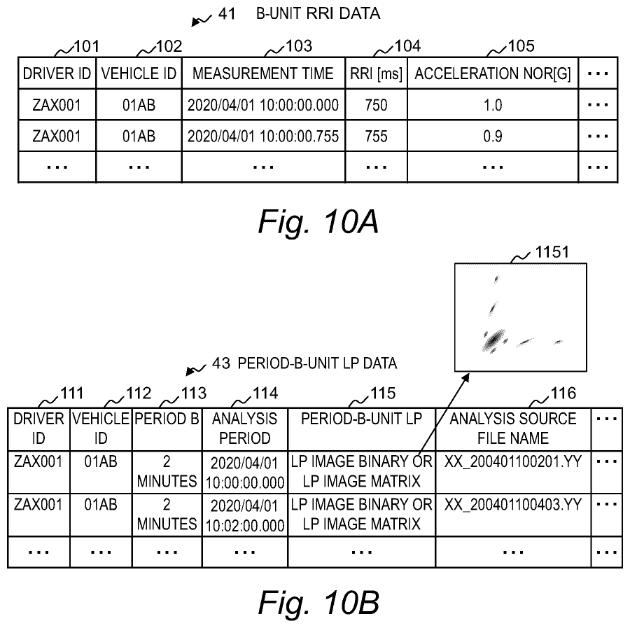

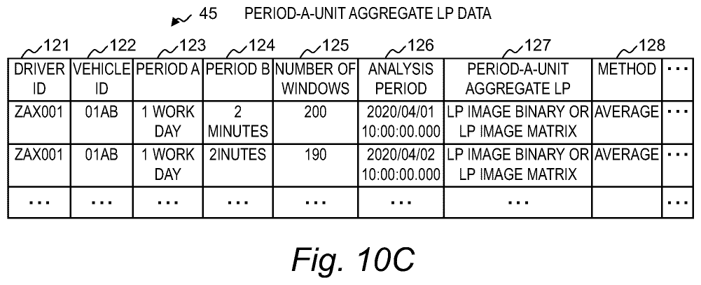

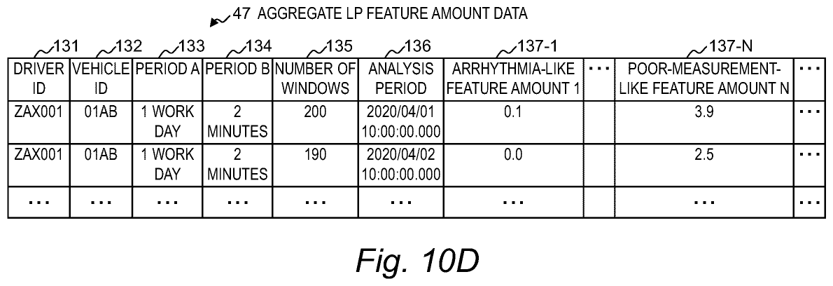

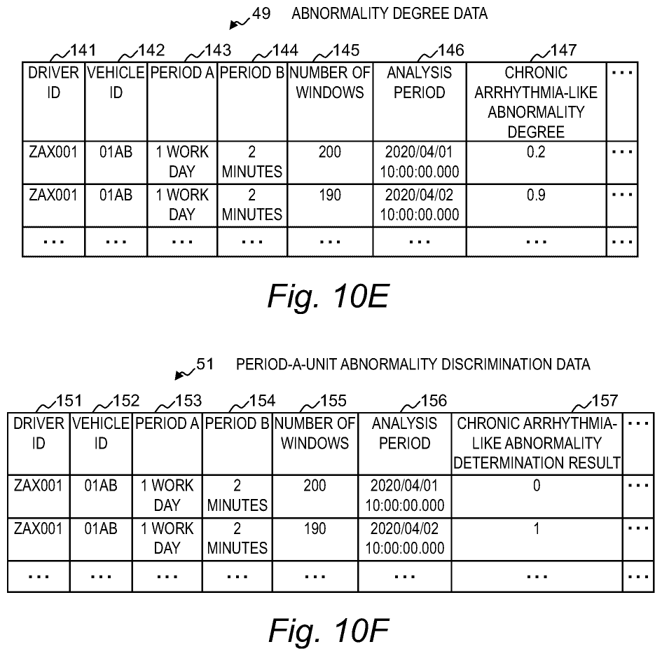

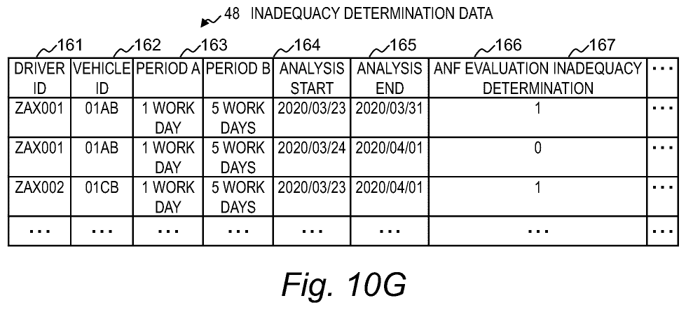

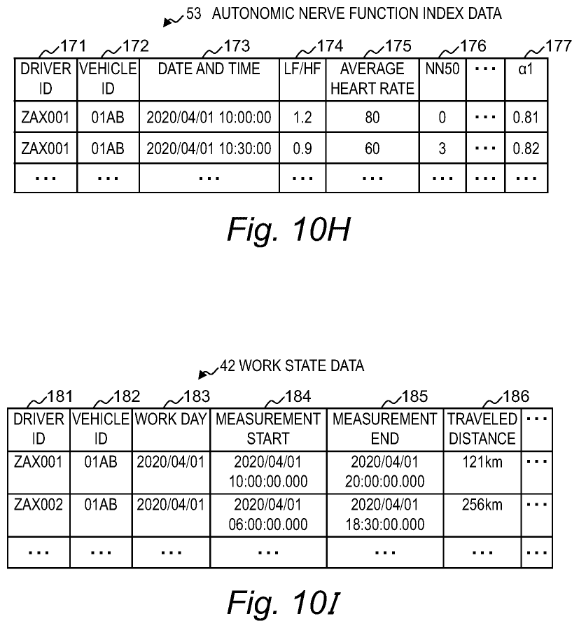

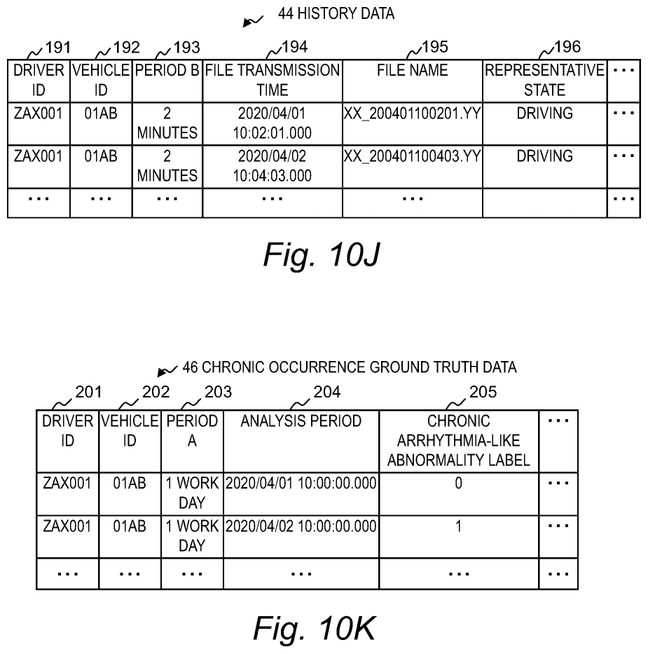

In regard to the abnormality degree calculated in this processing, for example, the discrimination probability of the presence or absence of the chronic occurrence, which is obtained by inputting the aggregate LP feature amount data 47 to the chronic occurrence discrimination model 52 as described above, is calculated as a chronic arrhythmia-like abnormality degree 147, which is shown in FIG. 10E, and can be used as the abnormality degree data 49.

Finally, the abnormal value chronic occurrence evaluation module 25 performs chronic occurrence discrimination processing (Step S32) through use of the aggregate LP feature amount data 47, the abnormality degree data 49, and the chronic occurrence discrimination model 52 to discriminate whether or not an arrhythmia-like abnormal value is chronically occurring during the period A, and stores a result thereof in the period-A-unit abnormality discrimination data 51.

The abnormal value chronic occurrence evaluation module 25 acquires the chronic arrhythmia-like abnormality degree 147 from the abnormality degree data 49 on a subject driver. When the chronic arrhythmia-like abnormality degree 147 exceeds an abnormality determination threshold value set in advance, the abnormal value chronic occurrence evaluation module 25 sets “1” as a chronic arrhythmia-like abnormality determination result 157, which is shown in FIG. 10F, of the period-A-unit abnormality discrimination data 51, and sets “0” when the chronic arrhythmia-like abnormality degree 147 is equal to or smaller than the abnormality determination threshold value.

As described with reference to FIG. 3, when the history data 44 is additionally read in the period B data reading processing (Step S23) and the period-A-unit aggregate LP is generated only from the period-B-unit RRI data 41 measured in the predetermined driver state, a model suitable for the predetermined driver state may be selected and used as the feature extraction model 50 for use in the aggregate LP feature amount generation processing (Step S26) and the chronic occurrence discrimination model 52 for use in the abnormality degree calculation processing (Step S31) and the chronic occurrence discrimination processing (Step S32). In this case, it is possible to take the measurement status of the RRI data into consideration to evaluate an occurrence status of an arrhythmia-like abnormal value with higher accuracy.

Further, in the first embodiment, the example in which the abnormality degree calculation processing (Step S31) and the chronic occurrence discrimination processing (Step S32) are sequentially performed has been described, but both may be performed simultaneously (in parallel). In this case, it suffices that the discrimination probability and the discrimination result, which have been obtained by inputting the aggregate LP feature amount data 47 to the chronic occurrence discrimination model 52, are stored as the abnormality degree data 49 and the period-A-unit abnormality discrimination data 51, respectively.

With the above, an effect that an LP for evaluating the degree of occurrence of an arrhythmia-like abnormal value during the period A can be generated even for the period-A-unit RRI data involving data lacking is obtained. It is also possible to discriminate whether or not the occurrence of the arrhythmia-like abnormal value is chronic.

Therefore, through use of the obtained period-A-unit abnormality discrimination data 51, it is possible to exclude an inadequate period from the period A even in an environment involving data lacking, thereby producing an effect of improving reliability of autonomic nerve function evaluation.

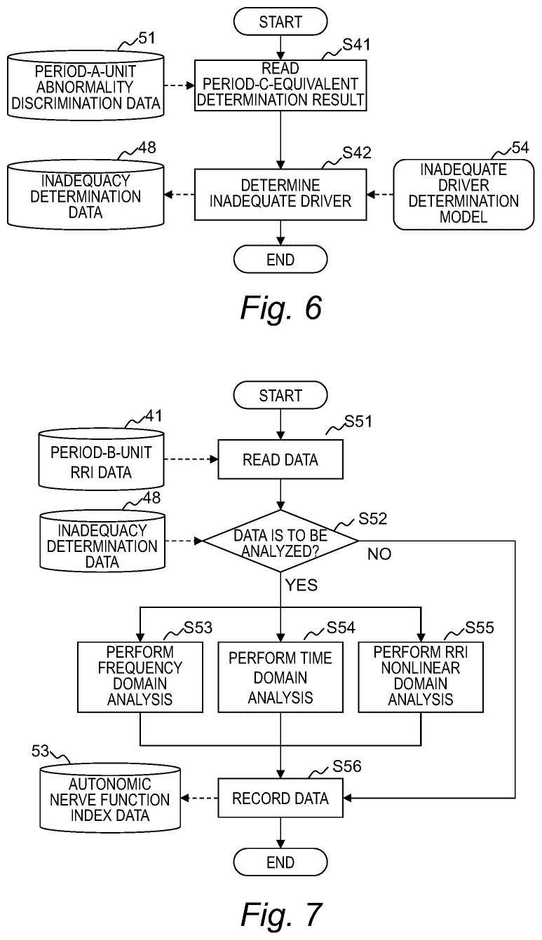

FIG. 6 is a flow chart for illustrating an example of processing for determining a driver inadequate for the autonomic nerve function evaluation, which is performed by the biometric data evaluation server 1. In the first embodiment, the example in which the period A is set to one work day is described, but the occurrence status of an arrhythmia-like abnormal value of the driver can vary depending on exercise stress and health condition.

It is thus difficult to determine all pieces of data on the driver to be inadequate for the autonomic nerve function evaluation from that point on based only on the chronic arrhythmia-like abnormality determination result 157 corresponding to the period A. In order to perform the above-mentioned determination, it is considered that it is desired to perform the evaluation by taking into consideration the chronic arrhythmia-like abnormality determination result 157 corresponding to the period C having a length equal to or longer than that of the period A.

Therefore, in processing for evaluating whether or not the driver is inadequate for the autonomic nerve function evaluation, the inadequate driver determination module 26 first performs period-C-equivalent determination result reading processing (Step S41) for the most recent period C by reading out the period-A-unit abnormality discrimination data 51 present within the period C.

Then, with the period-A-unit abnormality discrimination data 51 within the period C being used as input, the inadequate driver determination module 26 causes the inadequate driver determination model 54 to calculate a determination result (ANF evaluation inadequacy determination 167 of FIG. 10G) of determining whether or not the driver is inadequate for the autonomic nerve function evaluation based on the RRI data, and stores the determination result in the inadequacy determination data 48 (Step S42).

As the ANF evaluation inadequacy determination 167 of the inadequacy determination data 48, “1” is set in a case of being inadequate for the autonomic nerve function evaluation, and “0” is set in a case of being usable for the autonomic nerve function evaluation.

For example, when the period A is set to one work day, the period C may be set to five work days. Examples to be used as the inadequate driver determination model 54 may include a statistical model for statistically setting a determination threshold value by comparing occurrence distributions of the ANF evaluation inadequacy determination 167 for the period A with the period C being used as the total between a case of steadily exhibiting the arrhythmia-like abnormal value and a case of accidentally exhibiting the arrhythmia-like abnormal value depending on conditions of the subject driver.

With the above, the inadequate driver determination module 26 can detect a driver considered to be steadily inadequate for the autonomic nerve function evaluation using the RRI data irrespective of changes in conditions of the subject driver such as the health condition and the exercise stress, thereby producing an effect of achieving the reliability of the autonomic nerve function evaluation.

FIG. 7 is a flow chart for illustrating an example of processing for calculating an autonomic nerve function index, which is performed by the biometric data evaluation server 1. In the first embodiment, an example of calculating the autonomic nerve function index through use of the RRI data having the same window width as the period B used for the LP generation is described. In other words, in the first embodiment, an example of calculating the autonomic nerve function index in units of an analysis window of two minutes is illustrated.

The processing for calculating the autonomic nerve function index is performed by a well known or publicly known method, and hence only an outline thereof is described below. First, the autonomic nerve function index calculation module 27 performs data reading processing for reading the period-B-unit RRI data 41 (Step S51).

Subsequently, in order to evaluate whether or not the driver is assumed to be steadily inadequate for the autonomic nerve function evaluation based on the RRI data, the autonomic nerve function index calculation module 27 determines adequacy or inadequacy of the analysis from the inadequacy determination data 48 (Step S52).

In this determination, the autonomic nerve function index calculation module 27 extracts a record in which the ANF evaluation inadequacy determination 167 is “0” from the inadequacy determination data 48, and uses the period-B-unit RRI data 41 corresponding to a period from an analysis start 165 to an analysis end 166 to calculate the autonomic nerve function index data 53 in Step S53 to Step S55. Thus, the autonomic nerve function index calculation module 27 can exclude a record in which the ANF evaluation inadequacy determination 167 is “1”.

When it is determined that the driver is not inadequate for the analysis, the autonomic nerve function index calculation module 27 calculates the autonomic nerve function index data 53 from the RRI data through analysis to be performed as required. Examples of the analysis to be performed as required include frequency domain analysis (Step S53), time domain analysis (Step S54), and RRI nonlinear domain analysis (Step S55).

In the frequency domain analysis (Step S53), the autonomic nerve function index calculation module 27 calculates a frequency domain index from an RRI time series through use of the power spectral density. The RRI time series is unevenly spaced time-series data, and hence a power spectral density PSD is calculated through use of an autoregressive model or a maximum entropy method after resampling at equal intervals by spline interpolation or the like, or calculated through use of a publicly known method such as a Lomb-Scargle method which allows use of unevenly spaced data.

The autonomic nerve function index calculation module 27 uses the calculated PSD to calculate, as the frequency domain indices of the autonomic nerve function index data 53, for example, an integrated value LF in a low frequency domain of 0.05 hertz to 0.15 hertz, an integrated value HF in a high frequency domain of 0.15 hertz to 0.40 hertz, TP being a sum of LF and HF, LF/HF obtained by dividing LF by HF, and LFnu obtained by dividing LF by TP and converting a quotient thereof into a percentage.

In the time domain analysis (Step S54), the autonomic nerve function index calculation module 27 calculates a time domain index by calculating statistics of the RRI time series and a ΔRRI time series being a difference series of adjacent RRIs.

For example, the autonomic nerve function index calculation module 27 calculates an average heart rate being a reciprocal of an average value of the RRI time series and an SDNN being a standard deviation of the RRI data. From the ΔRRI time series, the autonomic nerve function index calculation module 27 also calculates, for example, NN50 being a total number of pieces of data in each of which an absolute value of a difference value forming the ΔRRI time series exceeds 50 milliseconds, pNN50 obtained by dividing NN50 by the total number of pieces of data in the ΔRRI time series, and an SDSD being a standard deviation of the ΔRRI, to thereby calculate results thereof as the time domain indices of the autonomic nerve function index data 53.

In the RRI nonlinear domain analysis (Step S55), the autonomic nerve function index calculation module 27 calculates nonlinear feature amounts by various methods. The autonomic nerve function index calculation module 27 calculates, for example, an elliptical area S by elliptical approximation of a region plotted as shown in FIG. 4A through LP analysis. The autonomic nerve function index calculation module 27 also calculates α1 and α2 by similar entropy or detrended fluctuation analysis and a tone and entropy based on tone-entropy analysis, to thereby calculate results thereof as RRI nonlinear domain indices of the autonomic nerve function index data 53.

After that, the autonomic nerve function index calculation module 27 collectively stores the calculated autonomic nerve function index group in the autonomic nerve function index data 53 (Step S56).

The autonomic nerve function index calculation module 27 may execute the above-mentioned processing steps in parallel or sequentially.

Meanwhile, in a case of the driver assumed to be inadequate for the analysis from the inadequacy determination data 48, the autonomic nerve function index calculation module 27 stores an inadequacy flag in the autonomic nerve function index data 53 without calculating the autonomic nerve function indices. The inadequacy flag may be stored in the autonomic nerve function index data 53 after the autonomic nerve function indices have been calculated.

With the above, it is possible to assign the inadequacy flag to the driver inadequate for the autonomic nerve function evaluation using the RRI data due to steady arrhythmia, thereby producing an effect of reducing a possibility of misinterpreting the obtained autonomic nerve function indices and improving the reliability of the autonomic nerve function evaluation.

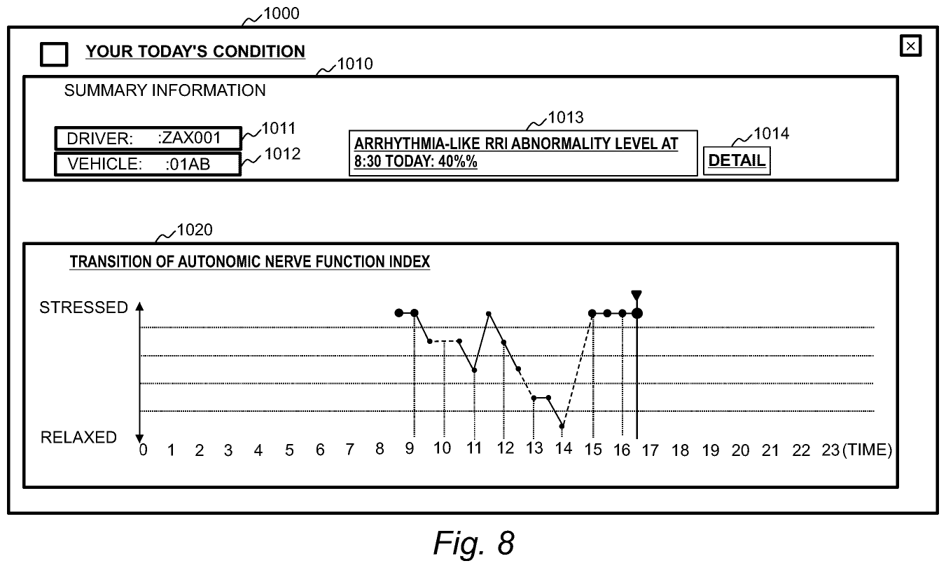

FIG. 8 is a diagram for illustrating an example of an autonomic nerve function evaluation result screen 1000, which is output by the result display module 28 to the display of the input-and-output device 5. When the biometric data evaluation server 1 has finished measuring the RRI data for the period A and generated the period-A-unit abnormality discrimination data 51, the biometric data evaluation server 1 causes the input-and-output device 5 to display the autonomic nerve function evaluation result screen 1000 for units of the period A.

On the autonomic nerve function evaluation result screen 1000, a driver ID 1011, a used vehicle (ID) 1012, and an arrhythmia-like RRI abnormality level 1013 are displayed in the upper part as summary information 1010 at the top. In addition, in the lower part, a transition of an autonomic nerve function index is displayed in five steps in 30-minute time series as a transition 1020 of the autonomic nerve function index. The autonomic nerve function index is an example that indicates a range from “relaxed” to “stressed.”

For example, at 10 o'clock within one work day in the period A, which is the subject of the first embodiment, data is lacking for some reason, and the autonomic nerve function index has failed to be calculated. In other words, this is a case in which a lack of RRI data has occurred during the period A and it is difficult to discriminate the degree of an arrhythmia-like abnormal value through application of the related-art LP.

However, in the first embodiment, the degree of an arrhythmia-like abnormal value can be evaluated even when a segment involving a lack is included in the period A. Therefore, work hours (for example, eight and half hours) for today and the degree of an arrhythmia-like abnormal value during the work hours are displayed in the arrhythmia-like RRI abnormality level 1013 of the summary information 1010 in the upper part.

For example, in the arrhythmia-like RRI abnormality level 1013, a value obtained by converting the discrimination probability recorded in the abnormality degree data 49 into a percentage representation may be displayed, to thereby quantitatively display the evaluation of the occurrence status of an arrhythmia-like abnormal value.

With the above, an effect that, even in a case in which a segment involving a lack of RRI data occurs in the period A, which can frequently occur during work, the driver can quantitatively understand whether or not the transition of the autonomic nerve function index displayed on the autonomic nerve function evaluation result screen 1000 is reliable enough is obtained.

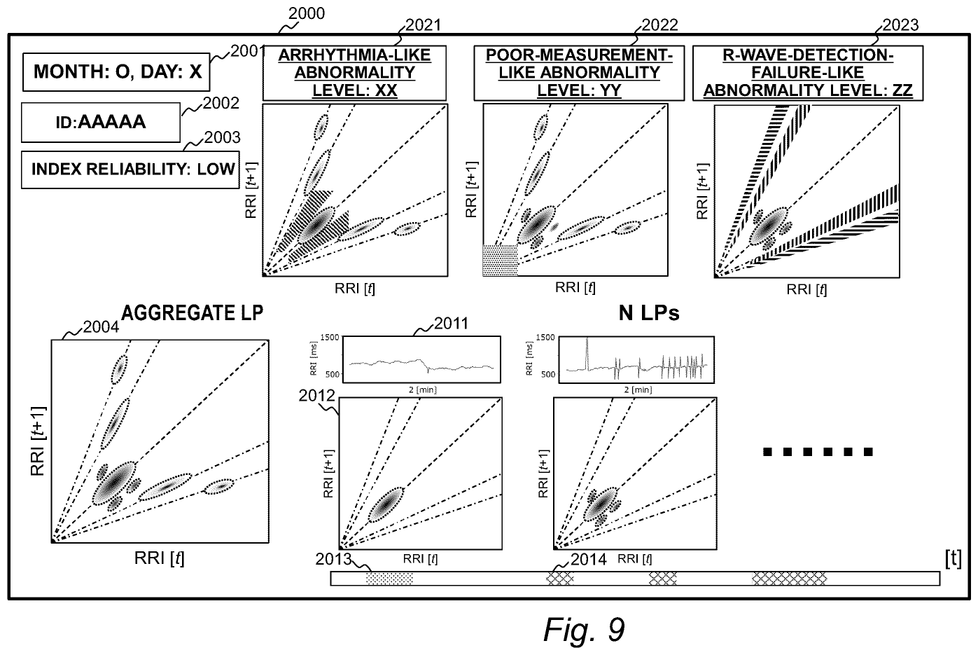

FIG. 9 is a diagram for illustrating an example of a detailed analysis screen 2000 for reliability of the RRI data measured during the period A, which is output by the result display module 28 to the display of the input-and-output device 5. When a detail button 1014 displayed in the summary information 1010 of the autonomic nerve function evaluation result screen 1000 illustrated in FIG. 8 is pressed, the input-and-output device 5 displays the detailed analysis screen 2000 regarding the measurement data for units of the period A.

In the upper left of the detailed analysis screen 2000, an applicable date 2001, a driver ID 2002, and reliability 2003 of the autonomic nerve function index for applicable one work day, which is the period A, are displayed.

In the lower left of the detailed analysis screen 2000, a period-A-unit aggregate LP 2004 is displayed. In the lower right, measured period-B-unit RRI data 2011 and period-B-unit LP data 2012 before having been aggregated in order to create the period-A-unit aggregate LP 2004 are displayed, and temporal changes of period-B-unit LP data can be examined by a scroll bar 2013 at the bottom.

Further, in the scroll bar 2013 at the lower part, a low-reliability segment 2014 is crosshatched in units of the period B. In the upper right of the detailed analysis screen 2000, a degree 2021 of an arrhythmia-like RRI abnormality and degrees 2022 and 2023 of poor-measurement-like RRI abnormalities are displayed together with grounds thereof.

In the first embodiment, for each of the arrhythmia-like, poor-measurement-like, and R-wave-detection-failure-like RRI abnormal values defined in the mask-pattern-based feature extraction method used for the feature extraction model 50, a region definition on the LP serving as grounds for the determination is superimposed on the period-A-unit aggregate LP 2004.

Each abnormality level of the period-A-unit aggregate LP 2004 can be displayed, for example, as a percentage by normalizing the feature amount relating to each mask by a maximum value of the feature amount. When a convolutional neural network is used as the feature extraction model 50, an attention map, which is means for visualizing grounds for determination thereof, may be displayed, to thereby visualize the grounds for the determination.

With the above, it is possible to easily present grounds for why the reliability of the autonomic nerve function evaluation ascribable to the arrhythmia-like abnormal value is decreasing by allowing a user or the like of the biometric data evaluation server 1 to refer to the detailed analysis screen 2000.

Further, in addition to the arrhythmia-like abnormal value, information relating to a status of a poor-measurement-like abnormal value can be presented, and the driver can determine whether or not the RRI data is normally measured. In addition, when the measurement status is found to be unsatisfactory only in some periods B, the driver can investigate a cause of the poor measurement in comparison with his or her own work knowledge.

### <Data Structure>

Next, a characteristic structure of each type of data to be used in the biometric data evaluation system is described.

FIG. 10A is a diagram for illustrating an example of a data structure of the period-B-unit RRI data 41 held in the storage device 4 of the biometric data evaluation server 1.

The period-B-unit RRI data 41 typically stores, in one record, a driver ID 101, a vehicle ID 102, a measurement time 103 of the RRI data, an RRI 104, and an acceleration norm 105.

The driver ID 101 stores the identifier of the driver acquired by the driver ID reading device 11 of the vehicle 7. The vehicle ID 102 stores the identifier of the vehicle 7 set in advance in the driving data collection device 10.

The measurement time 103 stores a date and time at which the heartbeat sensor 14 measured the RRI data. The RRI 104 stores a value (millisecond) detected by the heartbeat sensor 14. The acceleration norm 105 stores a vector value of an acceleration detected by the acceleration sensor 15.

FIG. 10B is a diagram for illustrating an example of a data structure of the period-B-unit LP data 43 held in the storage device 4 of the biometric data evaluation server 1. The period-B-unit LP data 43 typically stores, in one record, a driver ID 111, a vehicle ID 112, a window width 113 of the period B, an analysis period 114, a period-B-unit LP 115, and an analysis source file name 116.

The driver ID 111 stores the identifier of the driver in the same manner as in FIG. 10A. The vehicle ID 112 stores the identifier of the vehicle 7 in the same manner as in FIG. 10A. The period B 113 stores a length of the period B. The analysis period 114 stores a date and time of a start point of the period B.

The period-B-unit LP 115 may store an array obtained by flattening two-dimensional LP matrix components in the period B. In another case, the period-B-unit LP 115 may store information obtained by binary-encoding an LP image obtained through conversion with each LP matrix component as the brightness. In another case, the period-B-unit LP 115 may store the LP matrix components in a direction of columns (or fields) that differ among the components. The analysis source file name 116 stores a file name (or path) of the period-B-unit LP data 43.

FIG. 10C is a diagram for illustrating an example of a data structure of the period-A-unit aggregate LP data 45 held in the storage device 4 of the biometric data evaluation server 1. The period-A-unit aggregate LP data 45 typically stores, in one record, a driver ID 121, a vehicle ID 122, a window width 123 of the period A, a window width 124 of the period B, the number 125 of windows indicating the number of segments of the period B in which data was actually present in the period A, an analysis period 126, and a period-A-unit aggregate LP 127, and a method indicating an aggregate method 128.

The driver ID 121 and the vehicle ID 122 store the identifier of the driver and the identifier of the vehicle 7, respectively, in the same manner as in FIG. 10A. The period A 123 stores a length of the period A. The period B 124 stores the length of the period B. The analysis period 126 stores a date and time of a start point of the period A. The period-A-unit aggregate LP 127 may store an array obtained by flattening two-dimensional LP matrix components in the period A. In another case, the period-A-unit aggregate LP 127 may store information obtained by binary-encoding an LP image obtained through conversion with each LP matrix component as the brightness. In another case, the period-A-unit aggregate LP 127 may store the LP matrix components in a direction of columns that differ among the components.

FIG. 10D is a diagram for illustrating an example of a data structure of the aggregate LP feature amount data 47 held in the storage device 4 of the biometric data evaluation server 1. The aggregate LP feature amount data 47 typically stores, in one record, a driver ID 131, a vehicle ID 132, a window width 133 of the period A, a window width 134 of the period B, the number 135 of windows indicating the number of segments of the period B that were actually present in the period A, an analysis period 136, and column names (for example, an arrhythmia-like feature amount 1 137-1 to a poor-measurement-like feature amount N 137-N) each indicating a characteristic name of the feature amount such as the feature amount being arrhythmia-like or poor-measurement-like regarding a feature amount group extracted from the period-A-unit aggregate LP.

The driver ID 131 to the analysis period 136 are the same as the driver ID 121 to the analysis period 126 of FIG. 10C.

The feature amount 1 to N (137-1 to 137-N) store, for example, the feature amount of the LP included in the poor-measurement-like mask Mask1 of FIG. 4B, the feature amount of the LP included in the PVC-like abnormal value mask Mask3, and the feature amount of the LP included in the missed one-beat detection mask Mask4 or the missed two-consecutive-beat detection mask Mask5.

FIG. 10E is a diagram for illustrating an example of a data structure of the abnormality degree data 49 held in the storage device 4 of the biometric data evaluation server 1. The abnormality degree data 49 typically stores, in one record, a driver ID 141, a vehicle ID 142, a window width 143 of the period A, a window width 144 of the period B, the number 145 of windows indicating the number of segments of the period B that were actually present in the period A, an analysis period 146, and the chronic arrhythmia-like abnormality degree 147.

The driver ID 141 to the analysis period 146 are the same as the driver ID 131 to the analysis period 136 of FIG. 10D. The chronic arrhythmia-like abnormality degree 147 stores, for example, the discrimination probability of the presence or absence of the chronic occurrence of arrhythmia, which have been obtained by inputting the aggregate LP feature amount data 47 to the chronic occurrence discrimination model 52.

FIG. 10F is a diagram for illustrating an example of a data structure of the period-A-unit abnormality discrimination data 51 held in the storage device 4 of the biometric data evaluation server 1. The period-A-unit abnormality discrimination data 51 typically stores, in one record, a driver ID 151, a vehicle ID 152, a window width 153 of the period A, a window width 154 of the period B, the number 155 of windows indicating the number of segments of the period B that were actually present in the period A, an analysis period 156, and the chronic arrhythmia-like abnormality determination result 157.

The driver ID 151 to the analysis period 156 are the same as the driver ID 141 to the analysis period 146 of FIG. 10E.

The chronic arrhythmia-like abnormality determination result 157 stores the value of “0” or “1” as a result of discriminating, by the abnormal value chronic occurrence evaluation module 25, whether or not an arrhythmia-like abnormal value is chronically occurring during the period A. When an arrhythmia-like abnormal value is chronically occurring, “1” is stored in the chronic arrhythmia-like abnormality determination result 157, and otherwise “0” is stored.

FIG. 10G is a diagram for illustrating an example of a data structure of the inadequacy determination data 48 held in the storage device 4 of the biometric data evaluation server 1. The inadequacy determination data 48 typically stores, in one record, a driver ID 161, a vehicle ID 162, a window width 163 of the period A, a window width 164 of the period C, an analysis start 165 of the period A at which the analysis was started, an analysis end 166 of the period A at which the analysis was ended, and the autonomic nerve function (shown as “ANF” in FIG. 10G) evaluation inadequacy determination 167 obtained through the evaluation regarding the period C.

The driver ID 161 to the period A 163 are the same as the driver ID 151 to the period A 153, respectively, of FIG. 10F. The period C 164 stores a period (window width) longer than the period A. The analysis start 165 and the analysis end 166 store a start date and time and end date and time of the period C.

In the autonomic nerve function (ANF) evaluation inadequacy determination 167, “1” is stored when the RRI data on the driver ID 161 is not adequate for the evaluation of the autonomic nerve function, and otherwise “0” is stored.

FIG. 10H is a diagram for illustrating an example of a data structure of the autonomic nerve function index data 53 held in the storage device 4 of the biometric data evaluation server 1. The autonomic nerve function index data 53 typically stores, in one record, a driver ID 171, a vehicle ID 172, and a date and time 173 as well as various autonomic nerve function indices calculated by the autonomic nerve function index calculation module 27.

The driver ID 171 and the vehicle ID 172 are the same as the driver ID 161 and the vehicle ID 162 of FIG. 10G. The date and time 173 stores the date and time at which the autonomic nerve function indices were calculated.

In the first embodiment, examples of the autonomic nerve function indices include LF/HF 174 being the frequency domain index, an average heart rate 175 being the time domain index, NN50 (176), and al being the RRI nonlinear domain index (177).

FIG. 10I is a diagram for illustrating an example of a data structure of the work state data 42 held in the storage device 4 of the biometric data evaluation server 1. The work state data 42 typically stores, in one record, a driver ID 181, a vehicle ID 182, a work day 183, a measurement start date and time 184, a measurement end date and time 185, a traveled distance 186, and the like.

The driver ID 181 and the vehicle ID 182 are the same as the driver ID 171 and the vehicle ID 172 of FIG. 10H. The work day 183 stores a date on which the driver worked. The measurement start date and time 184 and the measurement end date and time 185 store a date and time at which the measurement of the biometric data was started and a date and time at which the measurement was ended, respectively. The traveled distance 186 stores a distance traveled by the driver on the work day 183.

FIG. 10J is a diagram for illustrating an example of a data structure of the history data 44 held in the storage device 4 of the biometric data evaluation server 1. The history data 44 typically stores, in one record, a driver ID 191, a vehicle ID 192, a window width 193 of the period B, a file transmission time 194 of the period-B-unit RRI data, a file name 195 of the received period-B-unit RRI data, a representative state 196, and the like.

The driver ID 191 and the vehicle ID 192 are the same as the driver ID 181 and the vehicle ID 182, respectively, of FIG. 101. The period B 193 stores the length of the period B. The file transmission time 194 stores a date and time at which the driving data collection device 10 of the vehicle 7 transmitted the RRI data. The file name 195 stores a file name (or path) of the RRI data.

The representative state 196 stores the state of the driver during the period B for which the period-B-unit RRI data 41 was measured.

FIG. 10K is a diagram for illustrating an example of a data structure of the chronic occurrence ground truth data 46 held in the storage device 4 of the biometric data evaluation server 1. The chronic occurrence ground truth data 46 typically stores, in one record, a driver ID 201, a vehicle ID 202, a window width 203 of the period A, an analysis period 204, and a chronic arrhythmia-like abnormality label 205 being a ground truth label indicating whether or not there is a chronic arrhythmia-like abnormality.

The driver ID 201 and the vehicle ID 202 are the same as the driver ID 191 and the vehicle ID 192, respectively, of FIG. 10J. The period A 203 stores the length of the period A. The analysis period 204 stores the start date and time of the period A. The chronic arrhythmia-like abnormality label 205 stores “1” when there is a chronic arrhythmia-like abnormality, and otherwise stores “0”.

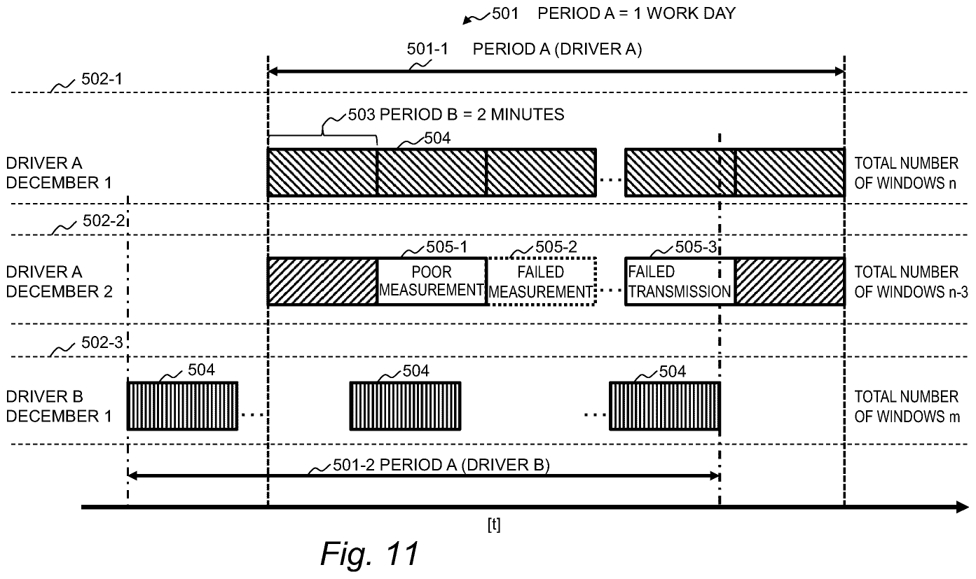

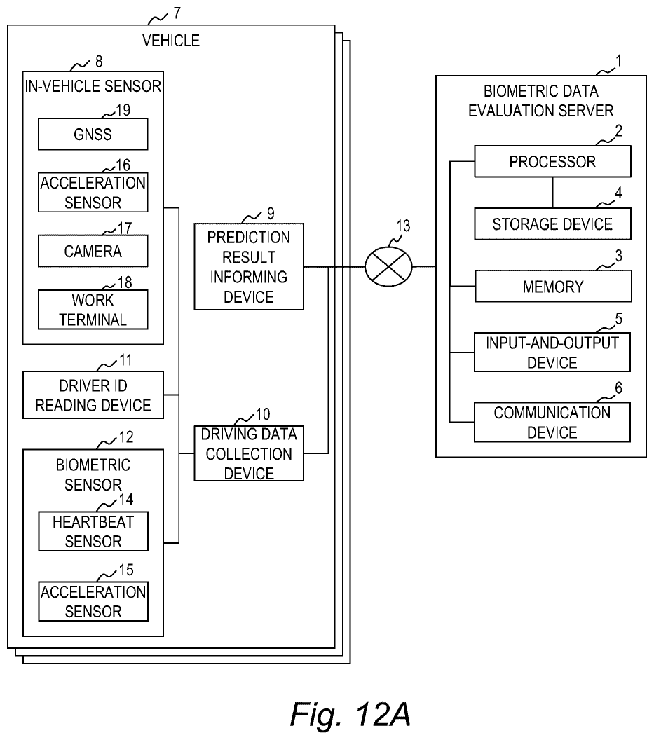

FIG. 11 is a diagram for illustrating an example relating to definitions of the period A and the period B and occurrence of data lacking. In the first embodiment, when a period A 501 is set to one work day (that is, a period from the start to end of work on a certain date) and a period B 503 is set to two minutes, periods A 501 (501-1 and 501-2) corresponding to three person-days and an example in which the period A 501 is divided in units of a period B 503 are described.

First, the period A 501 and period B 503 in a case in which no data lacking occurs are described. In a case of a driver A 502-1 on December 1, there are as many pieces of period-B-unit RRI data 504 obtained by dividing the period defined by the period A (driver A) 501-1 in units of the period B 503 as the number n of windows.

Meanwhile, in a case of a driver B 502-3 on December 1, the start time and end time of work differs from those of the driver A 502-1 on December 1, and the pieces of period-B-unit RRI data 504 are measured over a period corresponding to the period A (driver B) 501-2 different from the period A (driver A) 501-1. As a result, the pieces of period-B-unit RRI data 504 having the number m of windows, which differs from the number n of windows present in the period A 501-1 of the driver A on December 1, are subjected to the analysis.

Next, a driver A 502-2 on December 2 in a case in which data lacking occurs is described. In this case, the driver is the same with no change in work system, and hence, for the driver A 502-2 as well, the period A 501 is defined by the period A (driver A) 501-1 in the same manner as the driver A 502-1.

Meanwhile, the pieces of period-B-unit RRI data 504 measured during the period A (driver A) 501-1 have data lacking due to various factors. For example, a certain piece of period-B-unit RRI data 505-1 has a considerably small amount of valid RRI data due to the poor measurement, and has been determined to have data lacking.

Further, there is an empty pieces of period-B-unit RRI data on the biometric data evaluation server 1 due to a failure in the RRI data measurement during the period B 503 corresponding to a piece of period-B-unit RRI data 505-2, and the piece of period-B-unit RRI data 505-2 has been determined to have data lacking.

Further, there is no period-B-unit RRI data 504 on the biometric data evaluation server 1 due to an external facer 73 such as communication failures, and the piece of period-B-unit RRI data 505-3 has been determined to have data lacking.

As a result, data is lacking in the three segments, and only n−3 pieces of period-B-unit RRI data were measured for the driver A 502-2 on December 2, the n−3 pieces being smaller than the total number n of pieces of period-B-unit RRI data for the driver A 502-1 on December 1 by three pieces of data. The factors of data lacking are not limited to those described above.

As described above, there occurs a case in which, even when the measurement of the RRI data is attempted over the period A, the actually measured RRI data amount varies in such a manner that n, n−3, and m pieces of period-B-unit RRI data 41 were measured during the period A.

On the assumption that the period-B-unit RRI data 41 read into the biometric data evaluation server 1 has a considerably small amount of valid RRI data or is empty, the subject period-B-unit RRI data 41 may be inspected to determine presence or absence of data lacking.

As described above, in the biometric data evaluation system in accordance with the first embodiment, the biometric data evaluation server 1 uses the period-B-unit LP data 43 group calculated from a period-B-unit RRI data 41 group to calculate the period-A-unit aggregate LP data 45 subjected to the aggregation processing in units of the period A, uses the feature extraction model 50 to calculate, from the period-A-unit aggregate LP data 45, the aggregate LP feature amount data 47 including the feature amount indicating the degree of occurrence of an arrhythmia-like abnormal value, and inputs the aggregate LP feature amount data 47 to the chronic occurrence discrimination model 52, to thereby discriminate whether or not an arrhythmia-like abnormal value is chronically occurring in units of the period A.

Thus, the biometric data evaluation server 1 in accordance with the first embodiment can generate, even from the RRI data involving data lacking, an aggregate LP to be used for evaluating the degree of an arrhythmia-like RRI abnormal value.

Further, the biometric data evaluation server 1 generates the aggregate LP feature amount data 47 including the feature amount indicating the degree of occurrence of an arrhythmia-like abnormal value from the aggregate LP, to thereby be able to quantitatively evaluate the degree of the occurrence of an arrhythmia-like abnormal value separately from the poor-measurement-like abnormal value and other RRI abnormal values ascribable to factors different from arrhythmia.

In addition, the biometric data evaluation server 1 uses the aggregate LP feature amount data 47, to thereby be able to discriminate whether or not an arrhythmia-like abnormal value is chronically occurring during the period A based on the RRI data corresponding to the period A involving data lacking.

With the above, an effect that, even when the RRI data is measured under a state that is not necessarily a resting state, such as during work involving driving of the vehicle 7, the biometric data evaluation server 1 can evaluate the reliability of the autonomic nerve function index obtained from the RRI data measured during the period A is obtained.

In the first embodiment described above, the example of applying this invention to the biometric data evaluation server 1 that manages the vehicle 7 has been described, but this invention is not limited thereto. For example, in place of the vehicle 7, this invention can be applied to a railway vehicle, a vessel, an aircraft, or another moving object that requires a driver or a pilot.

Further, for example, the subjects are not only drivers but also may be general employees engaged in work in an environment in which biometric data is remotely measured and people belonging to an environment in which biometric data is remotely measured.

Further, in the first embodiment described above, the example of generating an LP in units of the period B has been described, but this invention is not limited thereto. An LP may be generated in real time from the RRI data received from the vehicle 7, and the LP generation may be ended in units of the period B.

### Second Embodiment

Next, a second embodiment of this invention is described. Except for differences described below, each of components of a biometric data evaluation system in accordance with the second embodiment has the same function as that of each of the components of the first embodiment which are denoted by the same reference symbols, and hence description thereof is omitted.

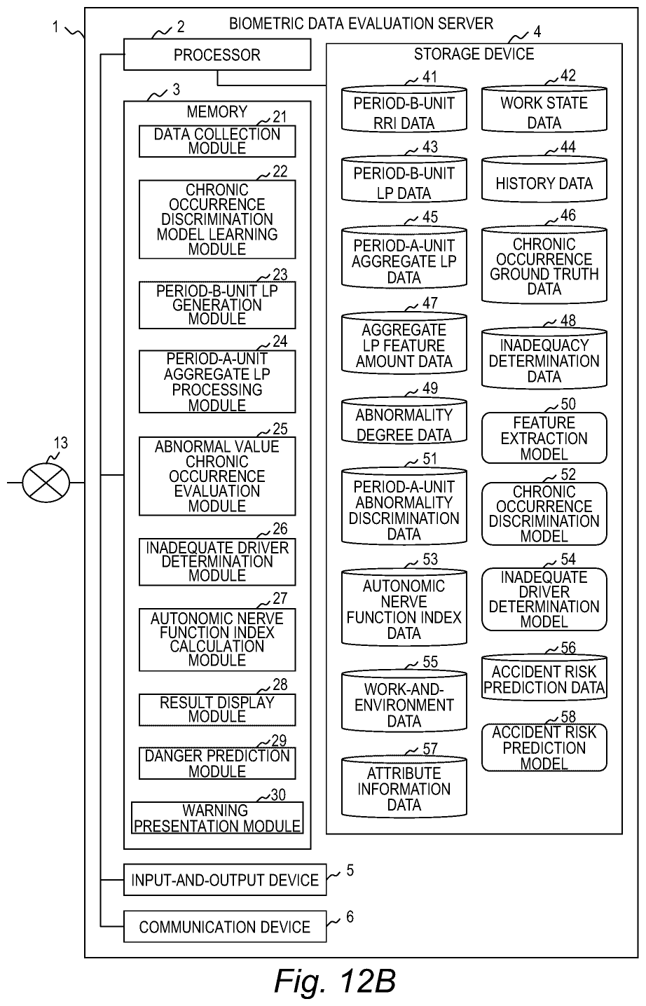

FIGS. 12A and 12B are block diagrams for illustrating an example of a main configuration of a biometric data evaluation system in accordance with the second embodiment of this invention in a case of also predicting a risk of a traffic accident or incident (hereinafter referred to as “accident risk”) for a driver through use of the autonomic nerve function index data 53.

In addition to the biometric sensor 12 that detects the biometric data on the driver, the driver ID reading device 11 that detects a driver ID for identifying the driver, and the driving data collection device 10 that collects the detected biometric data and the driver ID and transmits the biometric data and the driver ID to the biometric data evaluation server 1, the vehicle 7 further includes an in-vehicle sensor 8 that detects a work state and a traveling state and a prediction result informing device 9 that receives a warning corresponding to the accident risk for the driver from the biometric data evaluation server 1 and presents the warning to the driver.

In the example of FIG. 12A, the driving data collection device 10, the prediction result informing device 9, and the driver ID reading device 11 are illustrated as separate devices, but can be formed of one mobile terminal. In this case, the mobile terminal functions as a driving data collection module, a prediction result informing module, and a driver ID reading module.

The in-vehicle sensor 8 can include a global navigation satellite system (GNSS) 19 that detects position information on the vehicle, an acceleration sensor 16 that detects a behavior and speed of the vehicle 7, a camera 17 that detects a traveling environment as a video, and a work terminal 18 that presents or records the work information on a boarding driver.

The in-vehicle sensor 8 is not limited to the above-mentioned example, and it is possible to use, for example, a ranging sensor that detects an object and/or a distance around the vehicle 7, a steering angle sensor that detects a driving operation, or an angular velocity sensor that detects a turning operation of the vehicle 7. In addition, the acceleration sensor is desired to be a triaxial acceleration sensor.

In addition to the data collection module 21, the chronic occurrence discrimination model learning module 22, the period-B-unit LP generation module 23, the period-A-unit aggregate LP processing module 24, the abnormal value chronic occurrence evaluation module 25, the inadequate driver determination module 26, the autonomic nerve function index calculation module 27, and the result display module 28, the memory 3 of the biometric data evaluation server 1 further loads, as a program, each of functional modules including a danger prediction module 29 and a warning presentation module 30. Each program is executed by the processor 2. Details of the respective functional modules are described later.

The storage device 4 of the biometric data evaluation server 1 stores data to be used by each of the above-mentioned functional modules. In addition to the period-B-unit RRI data 41, the period-B-unit LP data 43, the period-A-unit aggregate LP data 45, the aggregate LP feature amount data 47, the abnormality degree data 49, the period-A-unit abnormality discrimination data 51, the autonomic nerve function index data 53, the work state data 42, the history data 44, the chronic occurrence ground truth data 46, the inadequacy determination data 48, the feature extraction model 50, the chronic occurrence discrimination model 52, and the inadequate driver determination model 54, the storage device 4 further stores work-and-environment data 55, attribute information data 57, accident risk prediction data 56, and accident risk prediction model 58.

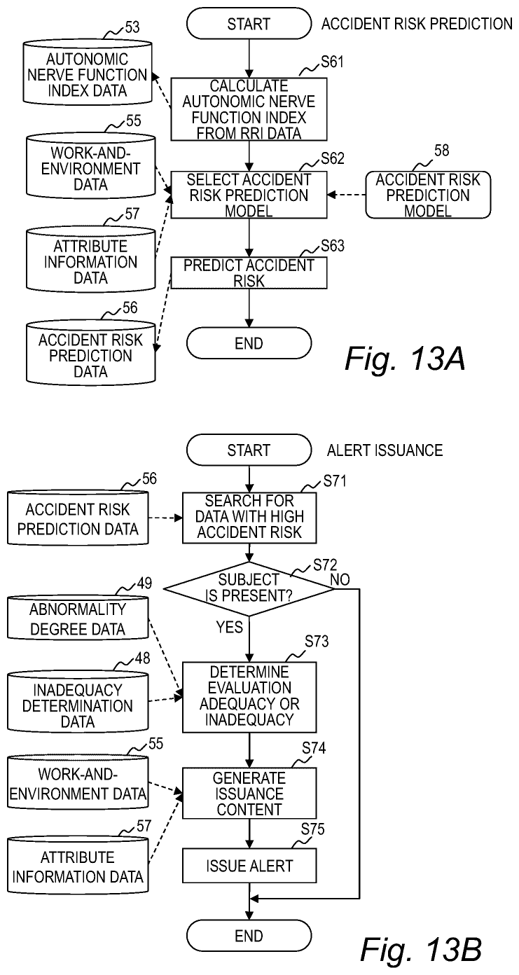

FIG. 13A is a flow chart for illustrating an example of processing for predicting the accident risk during work, which is performed by the biometric data evaluation server 1. This processing can be executed when the biometric data is received from the vehicle 7.

The danger prediction module 29 first inputs the RRI data acquired from the vehicle 7 to the autonomic nerve function index calculation module 27 to calculate the autonomic nerve function index data 53 (Step S61).

As described above, the autonomic nerve function index calculation module 27 calculates the power spectral density PSD in the frequency domain analysis to calculate LF/HF and LFnu as the frequency domain indices of the autonomic nerve function index data 53.

After that, the danger prediction module 29 selects and reads the accident risk prediction model 58 suitable for the driver of the traveling vehicle 7 (Step S62).

The accident risk prediction model 58 is a well known or publicly known machine learning model that uses the autonomic nerve function index data 53 on the driver of the traveling vehicle 7 as input to predict and output the accident risk after a predetermined time period, and is a model trained in advance.

As the accident risk prediction model 58, it is preferred that a plurality of models be prepared in advance depending on the work-and-environment data 55 that stores types of accident risks, traveling environments measured by the in-vehicle sensor 8 of the vehicle 7, predetermined work times, and the like. Having generated a plurality of models, it is possible to selectively use a suitable model in accident risk prediction processing (Step S63).

In addition, a plurality of models may be prepared depending on the attribute information data 57 that stores work characteristics (general road traveling, high-speed traveling, working continuously day and night, and the like), driving experience (years of driving, driving skills, and type of license being held), and health characteristics (gender, amount of sleep, and the like) of the driver. When a plurality of models have been generated in advance, a suitable accident risk prediction model 58 is selected based on the work-and-environment data 55 and the attribute information data 57 (Step S62).

In accident risk prediction model selection (Step S62), a plurality of accident risk prediction models 58 may be selected instead of selection of only a single model. In this case, it is desired to provide the accident risk prediction data 56 with a sign that can identify the accident risk prediction model 58 used for the prediction.

Finally, through use of the selected accident risk prediction model 58, the danger prediction module 29 inputs thereto the autonomic nerve function index data 53 to predict the accident risk after the predetermined time period, and stores the accident risk in the accident risk prediction data 56. The accident risk prediction data 56 can be calculated as the occurrence probability of an incident or accident.

FIG. 13B is a flow chart for illustrating an example of processing for issuing an alert for warning about an increase in accident risk, which is performed by the biometric data evaluation server 1. This processing is processing performed in Step S63 of FIG. 13A.

The warning presentation module 30 performs processing (Step S71) for searching the accident risk prediction data 56 as to whether or not data with a high accident risk to be subjected to alert issuance has been accumulated. The warning presentation module 30 can determine data having the occurrence probability of an incident or accident exceeding a predetermined threshold value as the data with a high accident risk.

The warning presentation module 30 determines whether or not the accident risk prediction data 56 to be subjected to the issuance is present (Step S72). When the accident risk prediction data 56 to be subjected to the issuance is present, the warning presentation module 30 advances the process to Step S73, and otherwise ends the processing.

Subsequently, the warning presentation module 30 performs evaluation adequacy-or-inadequacy determination processing (Step S73) to determine whether or not there is any concern about the evaluation of the autonomic nerve function index that has been input to the accident risk prediction model 58. In the evaluation adequacy-or-inadequacy determination processing (Step S73), for example, it is determined based on the inadequacy determination data 48 whether or not the driver is steadily causing an arrhythmia-like abnormal value even on different work days and there is a possibility of erroneous issuance.

Further, for example, the warning presentation module 30 determines based on the abnormality degree data 49 whether or not there is a possibility that the RRI data on the driver has been greatly affected by poor measurement at a time of the measurement. As the determination of the poor measurement, for example, it is possible to generate an LP from the RRI data read by the warning presentation module 30 and determine the poor measurement when a data amount (density) included in the poor-measurement-like mask Mask1 of FIG. 4B, the missed one-beat detection mask Mask4, or the missed two-consecutive-beat detection mask Mask5 exceeds a predetermined threshold value.

After that, the warning presentation module 30 generates issuance content for warning about an increase in accident risk corresponding to circumstances based on the work-and-environment data 55, and the attribute information data 57, and the accident risk prediction data 56 for the driver (Step S74), and issues the issuance content to the driver as an alert (Step S75).

In this case, the warning presentation module 30 acquires the vehicle ID of a transmission target from the driver ID based on the work-and-environment data 55 to identify the transmission target. When a driver manager is set as the transmission target in place of the driver, the warning presentation module 30 may set the transmission target to the input-and-output device 5 of the biometric data evaluation server 1 instead.

With the above-mentioned processing, the warning is transmitted to the vehicle of the driver for which the increase in accident risk has been detected. On the vehicle 7 that has received the warning, the prediction result informing device 9 notifies the driver of the warning. Meanwhile, on the biometric data evaluation server 1, the warning presentation module 30 displays the vehicle 7 to which the warning has been transmitted on the display of the input-and-output device 5.

In the second embodiment, an example in which the evaluation adequacy-or-inadequacy determination processing (Step S73) is performed and then notification is performed irrespective of a determination result thereof has been described, but it is not required to perform the alert issuance (Step S75) depending on the determination result. For example, when an arrhythmia-like abnormal value is steadily occurring even on different work days and a detected event with a high accident risk is an event that is significantly affected thereby, it is not required to issue the alert (Step S75) to the driver who has been determined to be inadequate for the autonomic nerve function evaluation. With the above, it is possible to inhibit reliability of the alert issuance from being lowered due to frequent erroneous issuance.

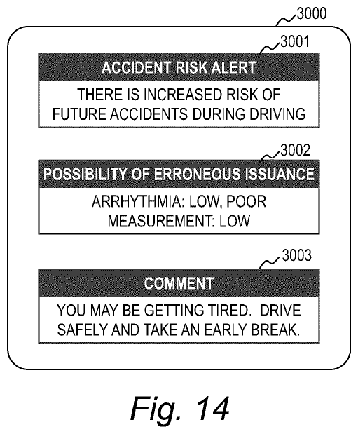

FIG. 14 is a diagram for illustrating an example of a warning presentation screen 3000 to be issued to the driver when an increase in accident risk is detected, which is output by the prediction result informing device 9 of the vehicle 7. The prediction result informing device 9 includes a display (not shown), and displays the warning presentation screen 3000 when the prediction result informing device 9 receives the warning from the biometric data evaluation server 1.

The warning presentation screen 3000 includes an area 3001 for displaying an accident risk alert and a comment area 3003 for displaying, for example, a countermeasure plan for eliminating an increase in accident risk as well as an area 3002 for displaying information relating to a possibility that the displayed issuance content is incorrect.

In the area 3001 for displaying the accident risk alert, for example, a warning message of an increased accident risk can be displayed. In addition, in the comment area 3003 on the warning presentation screen 3000, for example, a specific countermeasure plan for eliminating the increase in accident risk is presented, to thereby allow the driver who has been presented with the warning to understand and act on the next action to be taken in order to resolve a dangerous state, instead of merely being warned.

In addition, in the area 3002 for notifying of an erroneous issuance possibility on the warning presentation screen 3000, information for notifying of the possibility of erroneous issuance is displayed based on the reliability of the autonomic nerve function index measured from the driver subjected to the issuance.

For example, whether or not an arrhythmia-like abnormal value inadequate for the autonomic nerve function evaluation based on the RRI data is chronically occurring can be notified as, for example, “arrhythmia: low.” In addition, whether or not a large amount of poor measurement usually occurs in the measurement of the RRI data on the driver can be notified as, for example, “poor measurement: low.”

With the above, the driver can determine whether the notified alert issuance has been caused by low reliability of the measurement data and the autonomic nerve function evaluation or the accident risk is truly increasing, thereby increasing the reliability for alert issuance content.

In the second embodiment, the example of using the warning presentation screen 3000 to present a warning has been described, but another method may be used to present a warning. For example, a warning may be presented in an audio format for mechanically reading aloud text having content equivalent to the content displayed on the warning presentation screen 3000.

As described above, in addition to the processing described in the first embodiment, the biometric data evaluation system in accordance with the second embodiment inputs the autonomic nerve function index data 53 calculated from the RRI data to the accident risk prediction model 58 to calculate the accident risk prediction data 56 after the predetermined time period, and when an increase in occurrence risk of an accident or incident has been detected from the accident risk prediction data 56, issues an alert to the driver in consideration of content of the abnormality degree data 49, the inadequacy determination data 48, and the period-A-unit abnormality discrimination data 51, to thereby warn about an increase in accident risk.

Thus, at a time of issuing an alert about an increase in accident risk based on the autonomic nerve function index data 53 calculated from the RRI data, the biometric data evaluation server 1 in accordance with the second embodiment can issue an alert, in consideration of the characteristics of the driver subjected to the issuance, regarding events that can be the factors of erroneous issuance such as arrhythmia and poor measurement of the RRI data.

As a result, it becomes possible to consider canceling the alert issuance depending on the possibility of erroneous issuance and to issue an alert including the possibility of erroneous issuance in the notification content as well. Therefore, in regard to the alert issuance based on the autonomic nerve function index data 53 calculated from the RRI data, which is performed by the biometric data evaluation server 1, erroneous issuance thereof is reduced and acceptability of a reason for the erroneous issuance is improved, thereby producing an effect of greatly improving the reliability of the issued alert.

This invention is not limited to the above-described embodiments but includes various modifications. The above-described embodiments are explained in details for better understanding of this invention and are not limited to those including all the configurations described above. A part of the configuration of one embodiment may be replaced with that of another embodiment; the configuration of one embodiment may be incorporated to the configuration of another embodiment. A part of the configuration of each embodiment may be added, deleted, or replaced by that of a different configuration.

The above-described configurations, functions, processing modules, and processing means, for all or a part of them, may be implemented by hardware: for example, by designing an integrated circuit, and may be implemented by software, which means that a processor interprets and executes programs providing the functions. The information of programs, tables, and files to implement the functions may be stored in a storage device such as a memory, a hard disk drive, or an SSD (a Solid State Drive), or a storage medium such as an IC card, or an SD card.

The drawings illustrate control lines and information lines as considered necessary for explanation but do not illustrate all control lines or information lines in the products. It can be considered that almost of all components are actually interconnected.

### <Conclusion>

As described above, the biometric data evaluation system in accordance with each of the above-mentioned embodiments can be configured as follows.

(1) A biometric data evaluation server for evaluating biometric data, the biometric data evaluation server including: a processor (2); a memory (3); a data collection module (21) configured to receive beat-to-beat interval equivalent data (period-B-unit RRI data 41) from the biometric data on a subject (driver); and a Lorenz plot generation module (period-B-unit LP generation module 23, period-A-unit aggregate LP processing module 24) configured to calculate a Lorenz plot (period-B-unit LP data 43) from the beat-to-beat interval equivalent data (41) at a predetermined period, and output the obtained Lorenz plots as an aggregate Lorenz plot (period-A-unit aggregate LP data 45).

In accordance with the above-mentioned configuration, when an LP is generated for each of the periods B in which no lack of RRI data occurs and the LPs are aggregated in units of the period A, the knowledge relating to the period B can be applied to the period A including a lack of RRI data.

(2) The biometric data evaluation server in accordance with the above-mentioned item (1), wherein the predetermined period includes a first period (period A) and a second period (period B) shorter than the first period (period A), and wherein the Lorenz plot generation module (23, 24) is configured to calculate the Lorenz plot (period-B-unit LP data 43) from the beat-to-beat interval equivalent data (41) in units of the second period (period B), subject the Lorenz plots (43) calculated in units of the second period (period B) to aggregation processing in units of the first period (period A) to calculate the aggregate Lorenz plot (45) corresponding to the first period (period A), and output the aggregate Lorenz plot (45) corresponding to the first period (period A).

In accordance with the above-mentioned configuration, when an LP is generated for each of the periods B in which no lack of RRI data occurs and the respective LPs are aggregated in units of the period A, the knowledge relating to the period B which has been hitherto obtained through research can be applied to the period A including a lack of RRI data.

(3) The biometric data evaluation server in accordance with the above-mentioned item (2), wherein the Lorenz plot generation module (23, 24) is configured to determine presence or absence of a lack in the beat-to-beat interval equivalent data (41) from the Lorenz plot (43) calculated in units of the second period (period B), and exclude a lacking portion of the beat-to-beat interval equivalent data (41) in units of the second period (period B), to thereby perform the aggregation processing to calculate the aggregate Lorenz plot (45) corresponding to the first period (period A).

In accordance with the above-mentioned configuration, in the period-A-unit aggregate LP generation processing (Step S25), the chronic occurrence discrimination model learning module 22 calls the period-A-unit aggregate LP processing module 24, and the period-A-unit aggregate LP processing module 24 can generate the period-A-unit aggregate LP data 45, which is an LP that quantifies the feature amount of the RRI data corresponding to the period A, by performing the aggregation processing (Step S25) on the period-B-unit LP data 43 group corresponding to the periods B that are present in the period A to be subjected to the evaluation, that is, are not data lacking periods.

(4) The biometric data evaluation server in accordance with the above-mentioned item (2), wherein the Lorenz plot generation module (23, 24) is configured to calculate, as the aggregation processing, statistics in units of the first period (period A) for each of matrix components of Lorenz plot matrices representing a plurality of the Lorenz plots (43) calculated in units of the second period (period B).

In accordance with the above-mentioned configuration, the period-A-unit aggregate LP processing module 24 generates the period-A-unit aggregate LP data 45 by performing the statistical processing such as the averaging processing on the period-B-unit LP data 43 group for each component of the LP matrix, to thereby be able to perform, when the period-B-unit LP data 43 having characteristics noticeably different from others is present within the period A, the aggregation processing in which an influence of data having different characteristics is reduced through the addition of the statistical processing.

(5) The biometric data evaluation server in accordance with the above-mentioned item (2), wherein the Lorenz plot generation module (23, 24) is configured to set the second period (period B) shorter than the first period (period A) and equal to or longer than a duration required for heart rate variability analysis.

In accordance with the above-mentioned configuration, the period-A-unit aggregate LP processing module 24 can generate the period-A-unit aggregate LP data 45 for executing the heart rate variability analysis from the period-B-unit LP data 43 corresponding to the periods B that are not data lacking periods.

(6) The biometric data evaluation server in accordance with the above-mentioned item (2), wherein the Lorenz plot generation module (23, 24) is configured to receive information on a duration of a lack in the beat-to-beat interval equivalent data (41), and determine the second period (period B) based on the information on the duration of the lack in the beat-to-beat interval equivalent data (41).

In accordance with the above-mentioned configuration, the period-B-unit LP generation module 23 determines the duration of the period B based on the information on the duration of the lacking or missing of the RRI data received from the input device or the like, to thereby be able to generate the period-B-unit LP data 43 corresponding to the measurement environment of the biometric data.

(7) The biometric data evaluation server in accordance with the above-mentioned item (2), further including an abnormal value chronic occurrence evaluation module (25) configured to calculate a feature amount in a region defined in advance on the aggregate Lorenz plot (45), calculate a degree of occurrence of an arrhythmia-like abnormal value as abnormality degree data based on the feature amount, and output the abnormality degree data.

In accordance with the above-mentioned configuration, the abnormal value chronic occurrence evaluation module 25 can calculate the discrimination probability of the presence or absence of the chronic occurrence, which is obtained by inputting the aggregate LP feature amount data 47 to the chronic occurrence discrimination model 52, as the chronic arrhythmia-like abnormality degree 147 to generate the abnormality degree data 49.

(8) The biometric data evaluation server in accordance with the above-mentioned item (7), further including an inadequate person determination module (inadequate driver determination module 26) configured to calculate an occurrence frequency of the arrhythmia-like abnormal value from the aggregate Lorenz plots (45) in a third period (period C) equal to or longer than the first period (period A), and determine whether the subject is adequate for autonomic nerve function evaluation.

In accordance with the above-mentioned configuration, the inadequate driver determination module 26 can detect a driver considered to be steadily inadequate for the autonomic nerve function evaluation using the RRI data irrespective of changes in conditions of the subject driver such as the health condition and the exercise stress, thereby producing an effect of improving the reliability of the autonomic nerve function evaluation.

(9) The biometric data evaluation server in accordance with the above-mentioned item (8), further including an autonomic nerve function index calculation module (27) configured to calculate an autonomic nerve function index based on the beat-to-beat interval equivalent data (41) on the subject adequate for the autonomic nerve function evaluation based on a result of the determination performed by the inadequate person determination module (26), and output the autonomic nerve function index.

In accordance with the above-mentioned configuration, the autonomic nerve function index calculation module 27 can assign the inadequacy flag to the driver inadequate for the autonomic nerve function evaluation using the RRI data due to steady arrhythmia, thereby producing an effect of reducing the possibility of misinterpreting the obtained autonomic nerve function indices and improving the reliability of the autonomic nerve function evaluation.

(10) The biometric data evaluation server in accordance with the above-mentioned item (9), further including a warning presentation module (30) configured to determine reliability of the autonomic nerve function index based on the arrhythmia-like abnormal value, and display the reliability of the autonomic nerve function index on an output device.

In accordance with the above-mentioned configuration, the warning presentation module can inhibit reliability of the alert issuance from being lowered due to frequent erroneous issuance.

(11) The biometric data evaluation server in accordance with the above-mentioned item (10), further including a danger prediction module (29) configured to calculate, as an accident risk, a probability that the subject is to cause one of an accident or an incident after a predetermined time period, based on the autonomic nerve function index, wherein the warning presentation module (30) is configured to issue an alert for warning about an increase in accident risk to the subject having the accident risk exceeding a predetermined threshold value.

In accordance with the above-mentioned configuration, the driver can determine whether the notified alert issuance has been caused by low reliability of the measurement data and the autonomic nerve function evaluation or the accident risk is truly increasing, thereby increasing the reliability for alert issuance content.

What is claimed is:

1. A biometric data evaluation server including a processor and a memory for evaluating biometric data, the biometric data evaluation server comprising:
a data collection module configured to receive beat-to-beat interval equivalent data from the biometric data on a subject; and
a Lorenz plot generation module configured to calculate a Lorenz plot from the beat-to-beat interval equivalent data at a predetermined period, and output the obtained Lorenz plots as an aggregate Lorenz plot.

2. The biometric data evaluation server in accordance with claim 1,
wherein the predetermined period includes a first period and a second period shorter than the first period, and
wherein the Lorenz plot generation module is configured to calculate the Lorenz plot from the beat-to-beat interval equivalent data in units of the second period, subject the Lorenz plots calculated in units of the second period to aggregation processing in units of the first period to calculate the aggregate Lorenz plot corresponding to the first period, and output the aggregate Lorenz plot corresponding to the first period.

3. The biometric data evaluation server in accordance with claim 2,
wherein the Lorenz plot generation module is configured to determine presence or absence of a lack in the beat-to-beat interval equivalent data from the Lorenz plot calculated in units of the second period, and exclude a lacking portion of the beat-to-beat interval equivalent data in units of the second period, to thereby perform the aggregation processing to calculate the aggregate Lorenz plot corresponding to the first period.

4. The biometric data evaluation server in accordance with claim 2,
wherein the Lorenz plot generation module is configured to calculate, as the aggregation processing, statistics in units of the first period for each of matrix components of Lorenz plot matrices representing a plurality of the Lorenz plots calculated in units of the second period.

5. The biometric data evaluation server in accordance with claim 2,
wherein the Lorenz plot generation module is configured to set the second period shorter than the first period and equal to or longer than a duration required for heart rate variability analysis.

6. The biometric data evaluation server in accordance with claim 2,
wherein the Lorenz plot generation module is configured to receive information on a duration of a lack in the beat-to-beat interval equivalent data, and determine the second period based on the information on the duration of the lack in the beat-to-beat interval equivalent data.

7. The biometric data evaluation server in accordance with claim 2, further comprising an abnormal value chronic occurrence evaluation module configured to calculate a feature amount in a region defined in advance on the aggregate Lorenz plot, calculate a degree of occurrence of an arrhythmia-like abnormal value as abnormality degree data based on the feature amount, and output the abnormality degree data.

8. The biometric data evaluation server in accordance with claim 7, further comprising an inadequate person determination module configured to calculate an occurrence frequency of the arrhythmia-like abnormal value from the aggregate Lorenz plots in a third period equal to or longer than the first period, and determine whether the subject is adequate for autonomic nerve function evaluation.

9. The biometric data evaluation server in accordance with claim 8, further comprising an autonomic nerve function index calculation module configured to calculate an autonomic nerve function index based on the beat-to-beat interval equivalent data on the subject adequate for the autonomic nerve function evaluation based on a result of the determination performed by the inadequate person determination module, and output the autonomic nerve function index.

10. The biometric data evaluation server in accordance with claim 9, further comprising a warning presentation module configured to determine reliability of the autonomic nerve function index based on the arrhythmia-like abnormal value, and display the reliability of the autonomic nerve function index on an output device.

11. The biometric data evaluation server in accordance with claim 10, further comprising a danger prediction module configured to calculate, as an accident risk, a probability that the subject is to cause one of an accident or an incident after a predetermined time period, based on the autonomic nerve function index,
wherein the warning presentation module is configured to issue an alert for warning about an increase in accident risk to the subject having the accident risk exceeding a predetermined threshold value.

12. A biometric data evaluation system for evaluating biometric data, the biometric data evaluation system comprising:
a biometric data evaluation server including a processor and a memory; and
a moving object including a biometric sensor,
wherein the moving object includes a data collection device configured to cause the biometric sensor to detect biometric data including beat-to-beat interval equivalent data from a subject, and transmit the biometric data to the biometric data evaluation server, and
wherein the biometric data evaluation server includes:
a data collection module configured to receive the biometric data, and receive the beat-to-beat interval equivalent data; and
a Lorenz plot generation module configured to calculate a Lorenz plot from the beat-to-beat interval equivalent data at a predetermined period, and output the obtained Lorenz plots as an aggregate Lorenz plot.

13. The biometric data evaluation system in accordance with claim 12,
wherein the predetermined period includes a first period and a second period shorter than the first period, and
wherein the Lorenz plot generation module is configured to calculate the Lorenz plot from the beat-to-beat interval equivalent data in units of the second period, aggregate the Lorenz plots calculated in units of the second period in units of the first period to calculate the aggregate Lorenz plot corresponding to the first period, and output the aggregate Lorenz plot corresponding to the first period.

14. A biometric data evaluation method performed by a computer including a processor and a memory to evaluate biometric data, the biometric data evaluation method comprising:
a data collection step of receiving, by the computer, beat-to-beat interval equivalent data from the biometric data on a subject; and
a Lorenz plot generation step of calculating, by the computer, a Lorenz plot from the beat-to-beat interval equivalent data at a predetermined period, and output the obtained Lorenz plots as an aggregate Lorenz plot.

15. The biometric data evaluation method in accordance with claim 14,
wherein the predetermined period includes a first period and a second period shorter than the first period, and
wherein the Lorenz plot generation step includes calculating the Lorenz plot from the beat-to-beat interval equivalent data in units of the second period, aggregating the Lorenz plots calculated in units of the second period in units of the first period to calculate the aggregate Lorenz plot corresponding to the first period, and outputting the aggregate Lorenz plot corresponding to the first period.
